(19) United States
(12) Patent Application Publication
(10) Pub. No.: US 2010/0055173 A1
(43) Pub. Date: Mar. 4, 2010

(54) RELEASE OF STATINS IN THE INTESTINE

(76) Inventors: Adel Penhasi, Holon (IL); Maxim Gomberg, Jerusalem (IL)

Correspondence Address: WINSTON & STRAWN LLP;PATENT DEPARTMENT, 1700 K STREET, N.W., WASHINGTON, DC, 20006, US

(21) Appl. No.: 12/445,016

(22) PCT Filed: Oct. 9, 2007
(86) PCT No.: PCT/IL07/01220

§ 371 Date: Nov. 11, 2009

Related U.S. Application Data
(60) Provisional application No. 60/850,255, filed on Oct. 10, 2006.

(51) Int. Cl. A61K 9/52; A61K 31/47; A61K 31/366; A61K 31/505; A61K 31/40; A61K 31/404; A61K 31/351; A61P 9/10

(52) U.S. Cl. 424/452; 514/312; 514/460; 514/275; 514/423; 514/419

(57) ABSTRACT

The present invention provides a controlled absorption formulation in which modified release of the active ingredient preferentially occurs in the lower gastrointestinal tract, including the colon. The formulation supports a significantly higher bioavailability of the active ingredient in the body of the subject than that can be achieved from the currently used conventional formulation, such that therapeutically significant plasma levels of statin are maintained for an extended period after administration. The formulation preferably features a core, a subcoat surrounding the core comprising at least one water soluble hydrophilic carrier and an outer coating. The core is optionally and preferably in the form of a tablet.

### FIELD OF THE INVENTION

The present invention relates to a formulation for the controlled absorption of a medication, and in particular, to a formulation for the delayed onset, modified release of HMG-CoA reductase inhibitors (statins) predominantly in the lower gastrointestinal (GI) tract.

### BACKGROUND OF THE INVENTION

Modified release formulations for oral administration of drugs are beneficial for a number of reasons. For example, they enable the patient to ingest the formulation less frequently, which may lead to increased patient compliance with the dosing regimen. They may also result in fewer side effects, as peaks and troughs of the level of the drug in the bloodstream of the patient may be decreased, leading to a more even drug level in the blood over a period of time. Such formulations may also provide a longer plateau concentration of the drug in the blood. The size and frequency of dosing is determined by the pharmacodynamic and pharmacokinetic properties of the drug. The slower the rate of absorption, the less the blood concentrations fluctuate within a dosing interval. This enables higher doses to be given less frequently. For drugs with relatively short half-lives, the use of modified-release products may maintain therapeutic concentrations over prolonged periods.

Currently, delayed onset, modified release drug delivery systems administered by the oral route are usually based on either a gel forming matrix or coated formulations, or the combination thereof.

A delayed onset drug delivery system should preferentially deliver drugs to any part of the lower GI tract, as a site for topical delivery and subsequent absorption of the drug. This concept relies on the fact that the retention time of the drug delivery system through the colon may be the longest as compared to other parts of gastrointestinal tract. Likewise, such a delivery system could also advantageously use the unique continuous absorption characterizing the colon, which results in flatter, more consistent concentration levels of the drug in blood. Such absorption, of course, can contribute significantly to reduction of the fluctuations in blood drug concentration thus preventing the side effects which may appear upon using either immediate or conventional controlled release formulations, thereby improving compliance

Many different types of delayed onset formulations for delivery to the colon are known in the art. These include pH-dependent delivery systems; pH-independent delivery systems, including systems depending on factors such as hydrolytic degradation, hydrolysis, enzymatic degradation, and physical degradation, such as dissolution; and time-dependent delivery systems. Time-dependent systems release their drug load after a preprogrammed time delay. To attain colonic release, the lag time should equal the time taken for the system to reach the colon. The small intestinal transit time is generally considered to be in the region of three to four hours.

The statins are a class of compounds which contain a moiety that can exist as either a 3-hydroxy lactone ring or as the corresponding open ring dihydroxy acid. The structural formulas of these and additional HMG-CoA reductase inhibitors are described in M. Yalpani, “Cholesterol Lowering Drugs”, Chemistry & Industry, pp. 85-89 (1996).

The statins are orally effective in the reduction of serum cholesterol levels, by competitively inhibiting 3-hydroxy-3-methylglutaryl coenzyme A (HMG CoA) reductase, and play an important role in primary and secondary prevention of ischemic heart disease and myocardial infarct.

The statins include natural fermentation products lovastatin (described in U.S. Pat. No. 4,231,938) and mevastatin (described in U.S. Pat. No. 3,671,523); as well as a variety of semi-synthetic and totally synthetic products, which include simvastatin (U.S. Pat. No. 4,444,784); pravastatin sodium salt (U.S. Pat. No. 4,346,227); fluvastatin sodium salt (U.S. Pat. No. 5,354,772); atorvastatin calcium salt (U.S. Pat. No. 5,273,995); and cerivastatin sodium salt (also known as rivastatin; U.S. Pat. No. 5,177,080).

An osmosis-controlled release formulation for a statin is taught in U.S. Pat. No. 5,916,595, to Andrx which comprises a core containing a water swellable polymer and an osmotic agent, a channeling agent and a water insoluble cellulose polymer. Water is drawn into the tablet, which expands to the point where the outer coating fails in one particular area to form a constricted opening which releases the internal contents of the tablet which contain the drug. Thereafter, the aqueous medium of the tablet shell continues to release the drug as it dissolves until the osmotic pressure inside the tablet shell equals that of the surrounding environment. At the late stages of the in vivo release, the tablet shell collapses and/or disintegrates completely in order to substantially release the remaining drug. Complete release occurs over a period of 4-30 h.

U.S. Pat. No. 5,882,682 to Merck teaches controlled delivery of simvastin from a core by use of a water insoluble coating which contains apertures. The release rate of the simvastatin is a function of the number and size of the apertures in the coating, and again is a slow, extended form of release.

U.S. Pat. No. 4,997,658 to Merck teaches a method for lowering plasma cholesterol by using a HMG-CoA reductase inhibitor in a sustained release manner over a period of 6-24 hours as a slow, extended form of release, thereby reducing the amount of HMG-CoA reductase inhibitor circulating in the bloodstream.

WO 01/34123 to Andrx teaches a controlled release dosage form for a drug which may include the statins, in which the release is gradual, and occurs at about 10 to about 32 hours after oral administration; again the drug emerges from the formulation in a slow, extended form of release. This dosage form is intended to provide a moderate level of plasma statin concentration, wherein the mean time to maximum plasma concentration of the drug is about 10 to 32 hours after oral administration. This application does not relate to the way by which a higher blood plasma concentration of the active material may be obtained after administration.

WO 04/021972 to Biovail discloses formulations which putatively decrease the concentration of lovastatin and simvastatin and their active metabolites in the systemic circulation and at the same time provide increased concentrations of these statins in the liver. The disclosure teaches extended release formulations as preferred over a burst release formulation, and the structure of the formulations taught may for example feature a number of compartments.

US Patent Application 2003/0176502 to Athapharma describes controlled-release formulations of pravastatin in the small intestine, thereby limiting systemic exposure of the body to pravastatin.

WO 01/32162 describes a method comprising administration of an HMG CoA reductase inhibitor in a slow-release formulation to the small intestine that provides a clinically effective level in the portal vein and liver, but less than that required to provide a clinically effective blood level in the peripheral circulation.

WO 00/33821 to BMS describes an enteric-coated pravastatin bead formulation. WO 98/15290 to Astra describes a sustained release formulation of fluvastatin. EP1036563 describes a delayed-release oral formulation of dihydroxy open acid statin.

A gastrointestinal controlled delivery system is disclosed in U.S. Pat. Nos. 5,840,332 and 6,703,044, neither of which relate to the use of those formulations for very poorly water soluble drugs in general and make no reference whatsoever to the statins in particular.

Various references teach the metabolism and pharmacokinetics of statins in the human body (see for example M. J. Garcia et al., Clinical Pharmacokinetics of Statins, Clin. Pharmacol. 2003, 25 (6): 457-481).

Simvastatin is administered as the inactive lactone prodrug that must be hydrolyzed in the plasma and liver to the beta-hydroxy acid form for pharmacological activity. Simvastatin is believed to be metabolized in the liver and intestine, at least by the enzyme CYP3A, considering the beta-hydroxy acid form as the drug, the major active metabolites are 6-beta-hydroxymethyl and 6-beta-hydroxy simvastatin, which retain approximately 40% and 50%, respectively, of HMG-CoA reductase activity. Absorption reaches 60% while the bioavailability of the beta-hydroxy acid form following oral administration of simvastatin is less than 5%.

The poor bioavailability of simvastatin is mainly attributed to its low solubility in gastrointestinal fluids, low permeability through the mucosal membrane, and extensive first-pass metabolism. Since simvastatin (as stated above) is believed to be a CYP3A4 substrate, simvastatin may be expected to undergo significant intestinal metabolism.

The above cited reference also teaches that about 87% of the absorbed dose of simvastatin undergoes hepatic metabolism. The activation of simvastatin is by carboxyesterase-mediated hydrolysis, which occurs to a slight extent in plasma and in a higher extent in the liver. Both the parent lactone and the acid forms are normally present in very small amounts in the plasma, due to a high hepatic extraction ratio.

Simvastatin and its active acid forms are highly bound to plasma proteins, primarily to albumin (more than 95%). More than 98% of simvastatin is protein bound versus 94.5% for the open hydroxyl acid form. As only unbound drug is assumed to be able to enter the tissues, the high protein binding and low plasma concentrations of simvastatin are in agreement with the low peripheral tissue exposure in humans.

Physicians' Desk Reference 58th edition, 2004, pages 2113-2118 teaches the metabolism, pharmacokinetics, pharmacodynamics and side effects of simvastatin, and is hereby incorporated by reference as if fully set forth herein.

WO 2006/054308 to some of the applicants of the present invention relates to stable pharmaceutical formulation comprising a pharmaceutically acceptable form of atorvastatin as active ingredient, and at least one major excipient selected from the group consisting of starch, pregelatinized starch or lactose or a combination thereof, and pharmaceutical formulation of Atorvastatin or any acceptable salt thereof free of any stabilizer. WO 2006/103661 to some of the applicants of the present invention is directed to delayed onset controlled release formulation in which controlled release of the active ingredient occurs preferentially in the lower gastrointestinal tract including the colon.

PCT/IL2005/000539 published as WO 2005/115380 to some of the applicants of the present invention discloses a delayed burst release oral formulation for localized release of a statin in the GI tract. That formulation comprises a core comprising a statin and a burst controlling agent and an outer coating comprising a water insoluble hydrophobic carrier and a water insoluble hydrophilic particulate matter. The particulate matter, which allows entry of liquid into the core, is preferably a hydrophilic yet water insoluble polymer.

PCT/IL05/001234, published as WO 2006/054307 to some of the applicants of the present invention, discloses a delayed onset, modified release formulation for delivery of statins to the GI tract including the lower GI tract and the colon, providing an increased bioavailability as measured by AUC of a statin and/or active forms of said statin, relative to that resulting from the administration of an equivalent dose of conventional immediate release formulations. The formulations taught in that disclosure provides a delayed onset burst release formulation for drug release of a statin in the gastrointestinal tract comprising a drug containing core surrounded by a coating that limits the access of liquid to the core thereby controlling the release of the drug from the core to the GI tract.

However, the delayed onset burst release formulations taught in hitherto known disclosures suffered from the disadvantage that a significant amount of the active ingredient in the core was retained by the burst release coating after the delayed release burst. The present invention overcomes this disadvantage in the previous delayed onset burst release formulations.

There remains an unmet need for formulations of statins with improved bioavailability and pharmacokinetics of a statin while minimizing side effects and reduced dosage.

### SUMMARY OF THE INVENTION

The present invention provides a delayed onset, modified release formulation, for delivery of statins to the lower GI tract and the colon, which provides improved bioavailability. The present invention overcomes the deficiencies of known formulations of statins by providing a controlled absorption formulation for once a day administration in which rapid release of the active ingredient preferably occurs in the lower GI tract including the colon. Alternatively, such release may occur in the small intestine. The formulation provides significant plasma levels of a statin or its metabolites that are maintained for an extended period after administration.

According to a first aspect, the formulation according to the present invention provides a drug delivery formulation for localized drug release of a statin in the gastrointestinal tract comprising a core, a subcoat surrounding the core comprising at least one water soluble hydrophilic carrier, and an outer coating over the core. According to one embodiment, the core is preferably in the form of a tablet. It is now disclosed that the tablets without the subcoat exhibit a dose dependent retention of the active ingredient on the outer layer after the burst release, however the tablets with the subcoat overcome this problem and show significantly lower retention altogether without a dose dependency.

In one embodiment, the formulation is a delayed burst release oral formulation for localized release of a statin or a pharmaceutically acceptable salt or ester thereof in the gastrointestinal tract of a subject, comprising: (a) a core comprising at least one statin, and at least one burst controlling agent, wherein the burst controlling agent is a water insoluble polymer; (b) a subcoat surrounding the core comprising at least one water soluble hydrophilic carrier; and (c) an outer coating over the core, the outer coating comprising a water insoluble hydrophobic carrier and a water insoluble hydrophilic particulate matter, the water insoluble hydrophilic particulate matter allowing entry of liquid into said core.

According to a preferred embodiment the present invention provides a formulation for release of a statin and/or a pharmaceutically acceptable salt and/or ester thereof mainly in the colon of a subject, comprising: (a) a core that comprises an effective amount of statin and/or a pharmaceutically acceptable salt and/or ester thereof wherein the core contains at least one burst controlling agent and at least one disintegrant, and wherein the core is formed as a compressed tablet; (b) a subcoat surrounding the core comprising at least one water soluble hydrophilic carrier; and (c) an outer coating over the core, the outer coating comprising a water insoluble hydrophobic carrier and water-insoluble but hydrophilic particulate matter, contained in the carrier, that forms channels in the outer coating material upon contact with the colon medium, wherein the channels imbibe liquid and cause the at least one burst controlling agent to burst the coating, thereby providing delayed burst release of the statin and/or a pharmaceutically acceptable salt and/or ester thereof after at least two hours providing pharmacologically effective blood levels over a period extending over at least about 12 hours.

Delay time is defined as the time period from administration to the release. The delay time can be controlled by parameters such as the thickness of the said outer coating, the weight fraction of the said hydrophilic water-insoluble particulate matter in the said outer coating, the particle size and particle size distribution of the said hydrophilic water-insoluble particulate matter in the said outer coating, the nature and molecular weight of the said water insoluble hydrophobic carrier in the said outer coating, the presence of surface active agents such as surfactant, wetting agents, emulsifying agents, dispersing agents, and the nature of the said core of the said compressed tablet. The delay time can be planned to be up to 8 hours.

According to other embodiments, the core may be selected from the group consisting of tablets, pellets, microparticles, agglomerates, pills, capsule or any other solid dosage form.

According to one embodiment the present invention provides a drug delivery formulation for localized drug release of a statin in the gastrointestinal tract comprising a core comprising at least one statin, wherein the core includes at least one release controlling agent and an outer coating over the core the outer coating comprising a polymer that erodes and/or is ruptured after a predetermined period of time post administration.

According to some embodiments, the outer coating retains less than 2% of the total amount of the statin in the formulation, as measured in vitro following fast disintegration of a split dosage form. According to other embodiments, the outer coating retains less than 1% of the total amount of the statin in the formulation, as measured in vitro following fast disintegration of a split dosage form.

According to some embodiments, the formulation of the invention provides enhanced bioavailability of an insoluble statin, a pharmaceutically acceptable salt or ester thereof, or an active form thereof, as measured by the AUC compared to a substantially similar dose of an immediate release formulation of said statin. According to other embodiments, the formulation of the invention provides enhanced absorption of a statin, a pharmaceutically acceptable salt or ester thereof, or an active form thereof, as measured by the AUC compared to a substantially similar dose of an immediate release formulation of said statin. According to further embodiments, the formulation of the invention provides comparable AUC of a reduced dose of statin compared to an immediate release formulation of said statin.

In one embodiment, the term “insoluble statin” refers to a statin having aqueous solubility in the range of 1-100 micrograms per ml. According to various embodiments the statin is selected from lovastatin, mevastatin simvastatin, pravastatin, fluvastatin, atorvastatin, pitavastatin, rosuvastatin, rivastatin and cerivastatin also known as rivastatin, and salts thereof. The dosage levels of the active ingredient may easily be determined by one of ordinary skill in the art. According to certain currently preferred embodiments the statin is selected from simvastatin, atorvastatin and lovastatin. In a particular embodiment, said statin is simvastatin.

According to a certain embodiments of the present invention, the composition comprises a core containing an insoluble statin, a burst controlling agent and a disintegrant, the core being covered by a coating which may comprise a pH dependent coating film, preferably an enteric coating; a combination of at least one water soluble polymer and at least one water insoluble polymer; a combination of at least one swellable polymer and at least one water insoluble polymer; a combination of at least a water soluble pore forming agent and at least one water insoluble polymer; at least one swellable gel forming polymer; at least one erodible polymer; a combination of at least one pH dependent polymer and at least one water insoluble polymer; or a two-layer coating comprising a rupturable outer layer and swellable inner layer.

According to various embodiments, the water soluble hydrophilic carrier of the subcoat is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropylmethyl cellulose phthalate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate, and any other pharmaceutically acceptable polymer that dissolves in phosphate buffer pH>5.5 or mixtures thereof.

According to a currently preferred embodiment said water-soluble hydrophilic carrier is polyvinyl pyrrolidone.

According to one embodiment the subcoat further comprises at least one water insoluble particulate matter. According to various embodiments, said water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon doxide and cross-linked polyacrylic acid.

According to a currently preferred embodiment said water insoluble particulate matter is microcrystalline cellulose.

According to one embodiment the subcoat comprises povidone and microcrystalline cellulose.

The burst-controlling agent preferably comprises a water insoluble polymer for controlling the rate of penetration of water into the core and raising the internal pressure (osmotic pressure) inside the core. Such a burst-controlling agent is preferably able to swell upon contact with liquid.

According to various embodiments, the water insoluble polymer of the core is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

According to specific embodiments, the cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof.

According to specific embodiments, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

According to certain currently preferred embodiments, the water insoluble polymer is calcium pectinate or microcrystalline cellulose or a combination thereof.

In a preferable embodiment, the core further comprises at least one disintegrant. According to specific embodiments, the disintegrant is selected from the group consisting of croscarmellose sodium, crospovidone (cross-linked PVP) sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, low substituted carboxymethylcellulose, low substituted hydroxylpropylcellulose magnesium aluminum silicate and a combination thereof. More preferably, the disintegrating agent is croscarmellose sodium. Some commercial superdisintegrants suitable for use in the present invention include, Ac-Di-Sol, Primojel, Explotab, and Crospovidone.

According to some embodiments, the core further comprises at least one of an absorption enhancer, a binder, a hardness enhancing agent, and another excipient. According to specific embodiments the binder is selected from the group consisting of Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Optionally and preferably, the core also includes a stabilizer. More preferably, the stabilizer comprises at least one or more of butyl hydroxyanisole, ascorbic acid and citric acid. Optionally, the core may further include at least one of a buffering agent and a preservative.

According to some embodiments, the core further comprises a wicking agent. Preferably, the wicking agent is selected from the group consisting of colloidal silicon dioxide, kaolin, titanium dioxide, fumed silicon dioxide, alumina, niacinamide, sodium lauryl sulfate, low molecular weight polyvinyl pyrrolidone, m-pyrol, bentonite, magnesium aluminum silicate, polyester, polyethylene, or mixtures thereof.

According to some embodiments, the core further comprises a filler. Preferably, the filler is selected from the group consisting of microcrystalline cellulose, starch, lactitol, lactose, a suitable inorganic calcium salt, sucrose, or a combination thereof. More preferably the filler is lactose monohydrate.

According to preferred embodiments of the present invention, the core further comprises an antioxidant. Preferably, the antioxidant is selected from the group consisting of 4,4 (2,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid.

According to a preferred embodiment, the core further comprises ascorbic acid, which has several hydroxyl and/or carboxylic acid groups, and is able to provide a supply of hydrogen for regeneration of the primary antioxidant, exerting a synergistic effect on the inactivated antioxidant free radical.

According to a currently most preferred embodiment, the primary antioxidant is BHA.

According to preferred embodiments of the present invention, the core further comprises a chelating agent. Preferably, the chelating agent is selected from the group consisting of Antioxidants, Dipotassium edentate, Disodium edentate, Edetate calcium disodium, Edetic acid, Fumaric acid, Malic acid, Maltol, Sodium edentate, Trisodium edetate.

According to some embodiments of the present invention, the core further comprises one or both of a chelator and a synergistic agent (sequestrate). Preferably, the sequestrate is selected from the group consisting of citric acid and ascorbic acid. Without wishing to be limited by a single hypothesis, chelating agents and sequestrates may optionally be differentiated as follows. A chelating agent, such as citric acid, is intended to help in chelation of trace quantities of metals thereby assisting to prevent the loss of the active ingredient(s), such as simvastatin, by oxidation. A sequestrate, such as ascorbic acid, optionally and preferably has several hydroxyl and/or carboxylic acid groups, which can provide a supply of hydrogen for regeneration of the inactivated antioxidant free radical. A sequestrate therefore preferably acts as a supplier of hydrogen for rejuvenation of the primary antioxidant. Therefore, the combination of both a chelator and a sequestrate is preferred to protect the active statin ingredient.

According to additional embodiments, the core further comprises a flow-regulating agent. Preferably, the flow-regulating agent includes at least one of colloidal silicon dioxide and aluminum silicate. Most preferably, the flow-regulating agent is colloidal silicon dioxide.

Preferably, the core further comprises a lubricant. More preferably, the lubricant is selected from the group consisting of stearate salts; stearic acid, corola oil, glyceryl palmitostearate, hydrogenated vegetable oil, magnesium oxide, mineral oil, poloxamer, polyethylene glycol, polyvinyl alchol, sodium benzoate, talc, sodium stearyl fumarate, compritol (glycerol behenate), and sodium lauryl sulfate (SLS) or a combination thereof. Most preferably, the lubricant is magnesium stearate.

In another embodiment, the water-insoluble hydrophobic carrier of the outer coating is selected from the group consisting of: a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, said polymer corresponding to USP/NF “Ammonio Methacrylate Copolymer Type A”; an ethylmethacrylate/chlorotrimethyl ammonium ethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”; a dimethylaminoethylmethacrylate/methylmethacrylate and butylmethacrylate copolymer; a copolymer based on neutral methacrylic acid esters and dimethylaminoethyl methacrylate esters wherein the polymer is cationic in the presence of acids; an ethylacrylate and methylacrylate/ethylmethacrylate; and a methyl methylacrylate copolymer, the copolymer being a neutral copolymer based on neutral methacrylic acid and acrylic acid esters, ethylcellulose, shellac, and waxes.

In a particular embodiment, said water-insoluble hydrophobic carrier is ethylcellulose.

In another embodiment, the water insoluble hydrophilic particular matter of the outer coating is selected from the group consisting of a water insoluble polysaccharide, a water insoluble cross-linked polysaccharide, a water insoluble polysaccharide metal salt including calcium pectinate, a water insoluble cross-linked protein, a water insoluble cross-linked peptide, water insoluble cross-linked gelatin, water insoluble cross-linked hydrolyzed gelatin, water insoluble cross-linked collagen, a water insoluble cross linked polyacrylic acid, a water insoluble cross-linked cellulose derivative, water insoluble cross-linked polyvinyl pyrrolidone, microcrystalline cellulose, insoluble starch, microcrystalline starch and any combination thereof.

In a particular embodiment, said water insoluble hydrophilic particular matter is microcrystalline cellulose.

Optionally, the outer coating further comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

Optionally, the outer coating further comprises a stiffening agent. More preferably, the stiffening agent is cetyl alcohol.

Optionally, the outer coating or the core or both further comprises at least one of a wetting agent, a suspending agent, and a dispersing agent, or a combination thereof. More preferably, the wetting agent is selected from the group consisting of poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, and docusate sodium. Also more preferably, the suspending agent is selected from the group consisting of alginic acid, bentonite, carbomer, carboxymethylcellulose, carboxymethylcellulose calcium, hydroxyethylcellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), povidone (PVP), propylene glycol alginate, sodium alginate, sorbitan fatty acid esters, and tragacanth. Most preferably, the dispersing agent is selected from the group consisting of poloxamer, polyoxyethylene sorbitan fatty acid esters (polysorbates) and sorbitan fatty acid esters.

In another currently preferred embodiment, the outer coating further comprises a surfactant. In a particular embodiment, the surfactant in the outer coating comprises sodium lauryl sulfate (SLS).

Optionally, the formulation may comprise an enteric coating disposed on the outer coating. The enteric coating is more preferably selected from the group consisting of cellulose acetate phthalate, hydroxy propyl methyl cellulose acetate succinate, poly(methacrylic acid, methyl methacrylate)1:1 and (Eudragit® L100), poly(methacrylic acid, ethyl acrylate)1:1 (Eudragit® L30D-55), hydroxypropylmethyl cellulose phthalate, polyvinyl acetate phthalatealginic acid and sodium alginate. In another particular embodiment, said enteric coating comprises a methacrylic acid copolymer. In another particular embodiment, said enteric coating further comprises a plasticizer.

According to other embodiments of the present invention, the coating comprises a combination of at least one water-soluble polymer and at least one water insoluble polymer. Optionally and preferably, the water-soluble polymer is selected from the group consisting of polyvinyl alcohol, polyvinylpyrrolidone (PVP), methylcellulose, hydroxypropylcellulose, hydroxypropylmethyl cellulose, or polyethylene glycol, carboxymethyl cellulose (sodium salt), hydroxyethyl cellulose, a water soluble gum, polysaccharide and/or mixtures thereof.

Optionally and preferably, the water insoluble polymer is selected from the group consisting of a podimethylaminoethylacrylate/ethylmethacrylate copolymer, an ethylmethacrylate/chlorotrimethylammoniumethyl methacrylate copolymer, a dimethylaminoethylmethacrylate/methylmethacrylate and butylmethacrylate copolymer, a copolymer based on neutral methacrylic acid esters and dimethylaminoethyl methacrylate esters, an ethylacrylate and methylacrylate/ethylmethacrylate and methyl methylacrylate copolymer, ethylcellulose, shellac, zein, and waxes, paraffin, cellulose acetate, cellulose propionate, cellulose acetate propionate, cellulose acetate butyrate, cellulose acetate phthalate, cellulose triacetate, poly(methyl methacrylate), poly(ethylmethacrylate), poly(butyl methacrylate), poly(isobutyl methacrylate), and poly(hexyl methacrylate), poly(isodecyl methacrylate), poly(lauryl methacrylate), poly(phenyl methacrylate), poly(methylacrylate), poly(isopropyl acrylate), poly(isobutyl acrylate) poly(octadecyl acrylate), poly(ethylene), poly(ethylene) low density, poly(ethylene) high density, poly(ethylene oxide), poly(ethyleneterephthalate), poly(vinyl isobutyl ether), poly(vinyl acetate), poly(vinyl chloride) and polyurethane, and/or mixtures thereof. More preferably, the water insoluble polymer is ethylcellulose.

According to other embodiments of the present invention, the coating comprises a two-layer coating comprising a rupturable outer layer and swellable inner layer. Preferably, the two-layer coating ruptures independently of said core. Optionally and preferably, the inner layer comprises a disintegrant.

In certain embodiments, the inner layer comprises at least one polymer being able to swell when contacted by water. More preferably, the at least one polymer is selected from the group consisting of hydroxypropylmethyl cellulose, high molecular weight of carboxymethyl cellulose, high molecular weight of hydroxypropyl cellulose, high molecular weight of hydroxyethyl cellulose, high molecular weight of hydroxymethyl cellulose, polyhydroxyethyl methacrylate, polyhydroxymethyl methacrylate, polyacrylic acid, carbopol, polycarbophil, gums, polysaccharides, modified polysaccharides, cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid. Most preferably, the cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof. Also most preferably, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

According to other optional embodiments of the present invention, the inner layer comprises a disintegrant embedded in a water soluble film forming polymer. According to optional but preferred embodiments of the present invention, the inner layer comprises a combination of a water soluble polymer forming a film matrix, and a swellable water insoluble polymer particulate embedded into said film matrix. According to optional embodiments of the present invention, the rupturable outer layer comprises a brittle polymer. According to optional but preferred embodiments of the present invention, the rupturable outer layer comprises at least one permeation-enhancing agent.

According to other embodiments of the present invention, the rupturable outer layer comprises a water insoluble polymer selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes.

Preferably, the water insoluble polymer comprises ethylcellulose.

In a particular embodiment, the subcoat surrounding the core comprises povidone and microcrystalline cellulose in a ratio 2:8 to 8:2, and the outer coating comprises microcrystalline cellulose and ethyl cellulose in a ratio 1:9 to 7:3, cetyl alcohol in amount 5 to 15% of the ethyl cellulose weight. Preferably, the outer coating further contains 3-8% sodium lauryl sulfate (SLS). The subcoat and outer coating each constitutes 0.5-5% (w/w) and 3-50% (w/w) of core respectively.

A specific non-limitative example is a tablet containing a core, a pre-coating and an outer coating, wherein the core comprises 6.67% simvastatin 6.33% lactose monohydrate, 5.07% microcrystalline cellulose PH 101, 0.02% butylhydroxyanisole, 0.83% citric acid, 1.67% ascorbic acid, 1% Povidone K30, 0.43% croscarmellose sodium, 2% colloidal silicone dioxide, 2% croscarmellose sodium, 73.38% microcrystalline cellulose PH 102, and 0.6% magnesium stearate; the precoating comprises 50% povidone K30 and 50% microcrystalline cellulose PH 101; and the outer coating comprises 54.8% microcrystalline cellulose PH 102, 36.5% ethyl cellulose, 5% sodium lauryl sulphate, and 3.7% cetyl alcohol.

In one embodiment, the in vivo blood plasma concentration of the statin and/or a pharmaceutically acceptable salt and/or ester thereof is controlled by a lag time, providing a controlled absorption of the statin and/or a pharmaceutically acceptable salt and/or ester thereof and/or related active forms. In one specific embodiment, the formulations of the present invention are characterized in that the in vivo blood plasma concentration of the statin or a pharmaceutically acceptable salt or ester thereof in the subject is substantially zero for at least about 1.5 hours after oral administration of the formulation. In another specific embodiment, the formulations of the present invention are characterized in that the in vivo blood plasma concentration of the statin or a pharmaceutically acceptable salt or ester thereof in the subject is substantially zero for at least about two hours after oral administration of the formulation. In another specific embodiment, the in vivo blood plasma concentration of the statin or a pharmaceutically acceptable salt or ester thereof in the subject is substantially zero for at least about three hours after oral administration of the formulation. In yet another specific embodiment, the in vivo blood plasma concentration of the statin or a pharmaceutically acceptable salt or ester thereof in the subject is substantially zero for at least about four hours after oral administration of the formulation. The term “substantially zero”, as used herein, means that the statin is either not detected in the blood, or only minor amounts of the statin are detected in the blood.

According to one embodiment, the delayed burst release formulation of the present invention provides an increased amount of a statin, a pharmaceutically acceptable salt or ester thereof, or an active form thereof to the circulation of a subject, compared to a substantially similar dose of a conventional immediate release formulation of the stain. As used herein, the term “substantially similar dose” means a dose which is either equivalent or is substantially similar, for example a difference of not more than about 25%. The term “increased amount” means that administration of the formulations of the present invention result in higher blood levels of the statins or their active metabolites (e.g., 10% higher, 20% higher, 50% higher 100% higher, 200% higher, 500% higher etc.), as compared with blood levels achieved by administration of conventional statin formulations. The levels of the statins can be measured by determining the plasma concentration of the statins as a function of time following administration of the formulation, as known to a person of skill in the art. As demonstrated herein, administration of several simvastatin formulations according to the present invention to subjects resulted in blood levels that were significantly higher than the blood levels achieved after administration of conventional formulations of these statins. Further, importantly, the blood levels were maintained for significantly longer time periods as compared with the conventional formulation. For example, blood levels can be maintained for at least about 6 hours, preferably for about 8 hours, about 10 hours, about 12 hours and most preferably for about 24 hours after the delayed burst release occurs.

According to an alternative embodiment, the delayed burst release formulation of the present invention provides enhanced bioavailability of a statin, a pharmaceutically acceptable salt or ester thereof, or an active form thereof in a subject, compared to a substantially similar dose of an immediate release formulation of the stain. The term “enhanced bioavailability” means that administration of the formulations of the present invention results in higher bioavailability of the statins or their active metabolites (e.g., 10% higher, 20% higher, 50% higher 100% higher, 200% higher, 500% higher etc.), as compared with the bioavailability achieved by administration of conventional statin formulations. Bioavailability can be measured for example by comparing the AUC values obtained after administration of the formulations, as known to a person of skill in the art. As demonstrated herein, administration of several simvastatin formulations according to the present invention to subjects resulted in AUC values that were more than two fold higher than the AUC values obtained after administration of conventional formulations of these statins. Further, the AUC values were maintained for significantly longer time periods as compared with the conventional formulation, for example for at least about 6 hours, preferably for about 8 hours, about 10 hours, about 12 hours and most preferably for about 24 hours after the delayed burst release occurs.

According to yet another alternative embodiment, the delayed burst release formulation of the present invention provides a therapeutically effective amount of a statin, a pharmaceutically acceptable salt or ester thereof, or an active form thereof into the circulation of a subject. The term “therapeutically effective amount” refers to an amount of the statin which will result in a therapeutic effect of the disease or condition being treated, for example high blood cholesterol.

The present invention represents an improvement over WO 2004/021972 to Biovail, as the Biovail application seeks to reduce the concentration of statins in the blood circulation. In contrast, the present invention provides an increased concentration of statins or active forms thereof in the blood circulation relative to the dose administered, thus resulting in the administration of relatively lower dose of a statin or active forms thereof in the formulation administered to the subject (patient), thereby potentially reducing side effects by decreasing the total dose of statin to which the body of the subject is exposed.

As explained above, the statins are a class of compounds which contain a moiety that can exist as either a 3-hydroxy lactone ring or as the corresponding open ring dihydroxy acid. Typically, the statins can be administered as the inactive lactone prodrugs that must be hydrolyzed in the plasma and liver to the beta-hydroxy acid form for pharmacological activity. In accordance with the present invention, the delayed burst release formulations described herein are capable of providing a therapeutically effective amount of the hydroxy acid metabolite of a statin or a pharmaceutically acceptable salt or ester thereof into the circulation of a subject. In another embodiment, the formulation releases the statin in the gastrointestinal tract, and provides clinically effective amounts of an active form metabolite of said statin into the circulation of the subject.

According to other preferred embodiments of the present invention, there is provided a formulation for administering a statin to a subject, featuring a relatively lower dose of said statin. By “relatively lower dose” it is meant a dose that provides at least the same or similar pharmaceutical and/or therapeutic effect (if not a greater effect) as a conventional dose of a statin, while featuring a lower amount of statin than the conventional dose of the statin. It should be noted that a similar principle may optionally be applied for dosage forms featuring a plurality of different statins.

In another embodiment, the formulation releases substantially no statin in vitro for at least about 2 hours to about 6 hours, preferably at least about 2 hours, more preferably at least about 3 hours, also more preferably at least about 4 hours, also more preferably at least about 5 hours and most preferably at least about 6 hours. In other embodiments, the formulation releases substantially no statin in vitro for at least about 1 hour, or, in other embodiments, for at least about 1.5 hours.

In another embodiment, said statin is present in a decreased dosage amount of up to about 60% as compared to an immediate release formulation of said statin, while providing a substantially similar lowering of LDL blood concentration as said immediate release formulation.

In another embodiment, the formulation is characterized in that at least about 60% of the statin is released in vitro about 1 hour after the delayed burst release occurs.

The core of the formulations of the present invention contains a statin, which is preferably selected from simvastatin, lovastatin, mevastatin, pravastatin, fluvastatin, atorvastatin, cerivastatin and pitavastatin or pharmaceutically acceptable salts, esters, metabolites, hydrates, polymorphs, or crystals thereof. According to one currently preferred embodiment the statin is simvastatin. According to another currently preferred embodiment the statin is pitavastatin. According to other preferred embodiments the statin is lovastatin or atorvastatin.

The term “statin” as used herein includes also pharmaceutically acceptable salts, esters, metabolites, hydrates, polymorphs, or crystals thereof, and includes both statins in the lactone form or in the corresponding open dihydroxy acid.

The term “simvastatin” includes simvastatin and pharmaceutically acceptable salts, esters, metabolites, hydrates, polymorphs, or crystals thereof, in the lactone form or in the corresponding open dihydroxy acid, as disclosed for example in U.S. Pat. No. 4,444,784, which is hereby incorporated by reference in its entirety as if fully set forth herein.

The term “lovastatin” includes lovastatin and pharmaceutically acceptable salts, esters, metabolites, hydrates, polymorphs, or crystals thereof, in the lactone form or in the corresponding open dihydroxy acid, as disclosed for example in U.S. Pat. No. 4,231,938, which is hereby incorporated by reference in its entirety as if fully set forth herein.

The term “mevastatin” includes mevastatin and pharmaceutically acceptable salts, esters, metabolites, hydrates, polymorphs, or crystals thereof, in the lactone form or in the corresponding open dihydroxy acid, as disclosed for example in U.S. Pat. No. 3,671,523, which is hereby incorporated by reference in its entirety as if fully set forth herein.

The term “pravastatin” includes pravastatin and pharmaceutically acceptable salts, esters, metabolites, hydrates, polymorphs, or crystals thereof, in the lactone form or in the corresponding open dihydroxy acid, as disclosed for example in U.S. Pat. No. 4,346,227, which is hereby incorporated by reference in its entirety as if fully set forth herein.

The term “fluvastatin” includes fluvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,354,772, which is hereby incorporated by reference in its entirety as if fully set forth herein.

The term “atorvastatin” includes atorvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,273,995, which is hereby incorporated by reference in its entirety as if fully set forth herein.

The term “rivastatin” includes rivastatin and pharmaceutically acceptable salts, esters, metabolites, hydrates, polymorphs, or crystals thereof, in the lactone form or in the corresponding open dihydroxy acid, as disclosed for example in U.S. Pat. No. 5,177,080, which is hereby incorporated by reference in its entirety as if fully set forth herein.

The term “pitavastatin” (“nisvastatin”) includes pitavastatin and pharmaceutically acceptable salts, esters, metabolites, hydrates, polymorphs, or crystals thereof, in the lactone form or in the corresponding open dihydroxy acid, as disclosed for example in U.S. Pat. No. 5,011,930, U.S. Pat. No. 5,872,130, U.S. Pat. No. 5,856,336, which are hereby incorporated by reference in their entirety as if fully set forth herein.

As used herein, the term “active form” refers to any form of a molecule that can function as an HMG-CoA reductase inhibitor including the active ingredient administered and any active derivative resulting from metabolism or otherwise obtained from the parent molecule that can act as an HMG-CoA reductase. For example in the case of simvastatin marketed under the tradename ZOCOR® the known active forms include α-hydroxyacid of simvastatin and its 6β-hydroxy, 6β-hydroxymethyl, and 6β-exomethylene derivatives. The term “metabolite”, as used herein, includes any active form of the statin as described herein.

Suitable pharmaceutically acceptable salts include but are not limited to inorganic salts such as, for example, sodium, potassium, ammonium, calcium, and the like.

In another aspect, there is provided a method for providing a therapeutically effective amount of a statin, a pharmaceutically acceptable salt or ester thereof or an active form thereof to a subject, comprising orally administering to the subject a delayed burst release formulation of the invention, as detailed above.

The doses of the statins to be used in the formulations of the present invention can be determined by a person of skill in the art, and will vary depending on the statin being used, the patient, and the condition being treated. Typical known therapeutic doses for each of the statins can be used as a guide to determine the appropriate dose to be used herein. As mentioned above, the formulations of the present invention preferably contain a reduced dose of the statin, as compared with the corresponding conventional formulation, preferably up to about 60% of the conventional dose for each statin.

In another aspect, there is provided a method for providing enhanced bioavailability of a statin, a pharmaceutically acceptable salt or ester thereof or an active form thereof to the circulation of a subject, as measured by the AUC compared to a substantially similar dose of an immediate release formulation of said statin, comprising orally administering to the subject a delayed burst release formulation of the invention, as detailed above.

In another aspect, the invention provides a method of providing a delayed fast release of a statin, a pharmaceutically acceptable salt or ester thereof or an active form thereof in the gastrointestinal tract of a subject, comprising orally administering to the subject a delayed burst release formulation of the invention, as detailed above.

### BRIEF DESCRIPTION OF THE DRAWINGS

The invention is herein described, by way of example only, with reference to the accompanying drawings, wherein:

FIG. 1 shows the retention of Simvastatin (%) as a finction of dosage (mg) in tablets coated with TCDS without pre-coating that underwent total disintegration.

FIG. 2 shows the retention of Simvastatin (%) in tablets coated with TCDS without precoating and with different pre-coating procedures as specified in the Examples.

FIG. 3 demonstrates the Simvastatin accumulative release (%) over time (h) from tablets with type 1 cores (including Simvastatin 8 mg and 2% colloidal silicon dioxide, core weight 250 mg) coated with coating type A (TCDS).

FIG. 4 demonstrates the Simvastatin accumulative release (%) over time (h) from tablets with type 2 cores (including Simvastatin 10 mg and 2% colloidal silicon dioxide, core weight 300 mg) coated with coating type A (TCDS).

FIG. 5 demonstrates the Simvastatin accumulative release (%) over time (h) from tablets with type 3 cores (including Simvastatin 16 mg and 2% colloidal silicon dioxide, core weight 300 mg) coated with coating type A (TCDS).

FIG. 6 demonstrates the Simvastatin accumulative release (%) over time (h) from tablets with type 4 cores (including Simvastatin 10 mg and 0.7% colloidal silicon dioxide, core weight 316 mg) coated with coating type B (pre-coating, then TCDS).

FIG. 7 demonstrates the Simvastatin accumulative release (%) over time (h) from tablets with type 4 cores (including Simvastatin 10 mg and 0.7% colloidal silicon dioxide, core weight 316 mg) coated with coating type C (TCDS with 5% SLS).

FIG. 8 demonstrates the Simvastatin accumulative release (%) over time (h) from tablets with type 5 cores (including Simvastatin 10 mg and 1.5% colloidal silicon dioxide, core weight 300 mg) coated with coating type A (TCDS).

FIG. 9 demonstrates the Simvastatin accumulative release (%) over time (h) from tablets with type 5 cores (including Simvastatin 10 mg and 1.5% colloidal silicon dioxide, core weight 300 mg) coated with coating type B (pre-coating, then TCDS).

FIG. 10A-B demonstrate the Simvastatin accumulative release (%) over time (h) from tablets with type 5 cores (including Simvastatin 10 mg and 1.5% colloidal silicon dioxide, core weight 300 mg) coated with coating type C (TCDS with 5% SLS).

FIG. 11 demonstrates the Simvastatin accumulative release (%) over time (h) from tablets with type 5 cores (including Simvastatin 10 mg and 1.5% colloidal silicon dioxide, core weight 300 mg) coated with coating type D (pre-coating, then TCDS with 5% SLS).

FIG. 12 demonstrates the Simvastatin accumulative release (%) over time (h) from tablets with type 6 cores (including Simvastatin 10 mg and 2% colloidal silicon dioxide, core weight 300 mg) coated with coating type D (pre-coating, then TCDS with 5% SLS).

FIG. 13 demonstrates the Simvastatin accumulative release (%) over time (h) from tablets with type 6 cores (including Simvastatin 10 mg and 2% colloidal silicon dioxide, core weight 300 mg) coated with coating type E (pre-coating, then TCDS with 5% SLS, then thermal curing at 60° C. for 16 hours).

FIG. 14 demonstrates the Simvastatin accumulative release (%) over time (h) from tablets with type 7 cores (including Simvastatin 10 mg, SLS 1.33% and 1.5% colloidal silicon dioxide, core weight 300 mg) coated with coating type A (TCDS).

FIG. 15 demonstrates the Simvastatin accumulative release (%) over time (h) from tablets with type 7 cores (including Simvastatin 10 mg, SLS 1.33% and 1.5% colloidal silicon dioxide, core weight 300 mg) coated with coating type B (Pre-coating, then TCDS).

FIG. 16 demonstrates the Simvastatin accumulative release (%) over time (h) from tablets with type 7 cores (including Simvastatin 10 mg, SLS 1.33% and 1.5% colloidal silicon dioxide, core weight 300 mg) coated with coating type C (TCDS with 5% SLS).

FIG. 17 demonstrates the Simvastatin accumulative release (%) over time (h) from tablets with type 8 cores (including Simvastatin 20 mg and 1.5% colloidal silicon dioxide, core weight 600 mg) coated with coating type A (TCDS).

FIG. 18 demonstrates the Simvastatin accumulative release (%) over time (h) from tablets with type 8 cores (including Simvastatin 20 mg and 1.5% colloidal silicon dioxide, core weight 600 mg) coated with coating type D (Pre-coating, then TCDS with 5% SLS).

FIG. 19 demonstrates the Simvastatin accumulative release (%) over time (h) from tablets with type 9 cores (including Simvastatin 20 mg, 10% crospovidone, without colloidal silicon dioxide, core weight 300 mg) coated with coating type A (TCDS).

FIG. 20 demonstrates the Simvastatin accumulative release (%) over time (h) from tablets with type 9 cores (including Simvastatin 20 mg, 10% crospovidone, without colloidal silicon dioxide, core weight 300 mg) coated with coating type B (pre-coating, then TCDS).

FIG. 21 demonstrates the Simvastatin accumulative release (%) over time (h) from tablets with type 10 cores (including Simvastatin 20 mg and 1.5% colloidal silicon dioxide, core weight 300 mg) coated with coating type D (pre-coating, then TCDS with 5% SLS).

FIG. 22 demonstrates the Simvastatin accumulative release (%) over time (h) from tablets with type 11 cores (including Simvastatin 20 mg and 2% colloidal silicon dioxide, core weight 300 mg) coated with coating type D (pre-coating, then TCDS with 5% SLS).

FIG. 23 demonstrates the Simvastatin accumulative release (%) over time (h) from tablets with type 11 cores (including Simvastatin 20 mg and 2% colloidal silicon dioxide, core weight 300 mg) coated with coating type E (pre-coating, then TCDS with 5% SLS, then thermal curing at 60° C. for 16 hours).

FIG. 24 illustrates mean plasma simvastatin concentration-time curves.

FIG. 25 illustrates mean plasma SHA concentration-time curves.

### DESCRIPTION OF THE PREFERRED EMBODIMENTS

The present invention provides a formulation for controlled absorption of a statin, adapted so as to provide a time-delayed, modified release in the colon or small intestine. The formulation supports a lag time between oral administration and release of the active ingredient, providing higher bioavailability and lower dosage as compared to the currently used formulation. The formulation of the present invention optionally features non pH-dependent release, although alternatively and preferably features pH-dependent release, as for example with an enteric film coat.

The formulation of the present invention therefore provides a delayed onset, modified release formulation for delivery of statins in the lower GI tract preferentially to the colon or small intestine, which provides higher blood levels of statin or its metabolites in the bloodstream in comparison to a conventional immediate release formulation. The bioavailability is shown to be higher than that of a known reference product. The formulations according to the present invention should result in fewer side effects, greater safety, efficacy, and patient compliance.

The formulation of the present invention preferably comprises a delayed onset, modified release formulation, which is not a delayed burst release or delayed immediate or fast release formulation. The release is designed to occur within a period of less than 8 hours following oral administration, preferably with selective absorption of the active agent in the lower GI tract.

The present invention overcomes the deficiencies of known formulations of statins by providing a controlled absorption formulation for once a day administration in which modified release of the active ingredient preferably occurs in the lower GI tract including the colon. Alternatively, such release may occur in the small intestine. The formulation provides significant plasma levels of a statin or its metabolites that are maintained for an extended period after administration.

Without wishing to be limited by a single hypothesis, the formulation of the present invention is believed to have preferential release of the drug in the lower GI tract, resulting in increased amount of a statin and its active hydroxyl acid forms than would have been formed if the drug were allowed to be absorbed into the bloodstream prior to reaching the appropriate section(s) of the intestine.

Local intestinal production of a greater amount of the active metabolite, probably through the activity of colonic natural flora, or via other metabolic routes, will further enhance the desired clinical effect and allow the achievement of intestinal drug levels of these metabolites that are unattainable by systemic or conventional oral delivery.

By using the formulation according to the present invention, which is preferably a modified release formulation, it may be possible to obtain increased production of active forms in the gut than that which can be obtained through carboxyesterase-mediated hydrolysis in the liver.

Further advantages of at least partial colonic delivery are that statins probably have greater solubility in the colon, and colon transit times are longer, resulting in increased time of exposure of the drug, and hence greater absorption.

Orally administered drugs or chemical agents that are processed to active forms in the intestinal environment can be administered to a patient who suffers from impaired liver function. Impaired liver function prevents or diminishes the normal hepatic metabolism of drugs to active metabolites. The increased production of active forms following administration of the formulations of the present invention is believed to reduce stress on the liver. The liver enzyme CYP3A4 is also present in the intestine, hence metabolism in the intestine can serve an alternative for metabolism in the liver for such drugs in these patients.

Another reason for delivering statin in the lower GI tract using the formulations of the present invention is to avoid high concentrations of CYP3A4, in which is largely present at a high concentration in the upper GI tract, and thereby to enable the release of statin to take place in the lower GI tract where the concentration of CYP3A4 is relatively poor. This process can increase the bioavailability of the statin.

A further reason for delivering statin in the lower GI tract using the formulations of the present invention is reduce the potential for interaction between drugs. This is in the light of the fact that many drugs may either induce or inhibit the activity of CYP3A4, and thus the bioavailability of statin may be affected.

One of the advantages of the present invention is that optionally a reduced dosage of a statin may be used in comparison to the presently available formulations, which may lead to the following beneficial effects:
1. Reduced liver side effects, such as a reduced level of transaminase for example (dose-related side effect).
2. Reduced incidence of rhabdomyolysis, muscle pain, and/or reduced level of CPK (dose-related side effect).
3. Reduced gastrointestinal effects including but not limited to nausea, dyspepsia, flatulence, and/or constipation (may be dose related side effects; however, the present invention is expected to be effective to reduce these side effects in any event, regardless of whether they are dose related).
4. Better tolerated multiple drug treatment in which at least one additional drug is metabolized by the liver.

A further advantage of the present invention is that a reduced food effect on the release may be obtained, since the formulation according to the present invention provides a release occurring predominantly in the lower gastrointestinal tract including the colon. Metabolism and absorption of orally administered drugs are commonly known to be affected by interactions with food. The formulation of the present invention is expected to be little affected or even unaffected by such interactions, since metabolism and absorption of the statin occurs in the intestine, optionally and preferably in the colon.

The term “statin” includes also pharmaceutically acceptable salts or esters thereof.

The term “modified release” preferably includes delayed burst release and optionally includes any type of delayed release.

The delivery system of the present invention provides a modified formulation comprising a statin for controlled delivery of the active ingredient to the gastrointestinal tract. The delivery system comprises a drug containing core surrounded by a coating that limits the access of liquid to the core thereby controlling the release of the drug from the core to the GI tract.

The formulation is optionally in the form of a coated tablet. Alternatively, the formulation may be in the form of a pellet, microparticles, agglomerate, capsule or any other solid dosage form.

The combination of the selected materials for the core, the subcoating and the outer layer, and the relative concentrations thereof, as well as the thickness of the core matrix the subcoating and outer layer, determine both the lag time, which is the time, post administration, when the release starts, as well as the rate of release of the drug.

In a first aspect of the present invention, there is provided delayed burst release oral formulation for localized release of a statin or a pharmaceutically acceptable salt or ester thereof in the gastrointestinal tract of a subject, comprising:
(a) a core comprising at least one statin, and at least one burst controlling agent, wherein the burst controlling agent is a water insoluble polymer;
(b) a subcoat surrounding the core comprising at least one water soluble hydrophilic carrier; and
(c) an outer coating over the core, the outer coating comprising a water insoluble hydrophobic carrier and a water insoluble hydrophilic particulate matter, the water insoluble hydrophilic particulate matter allowing entry of liquid into said core.

### Subcoating

According to the present invention the subcoat surrounding the core comprises at least one water soluble hydrophilic carrier. According to various embodiments, the water soluble hydrophilic carrier is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropylmethyl cellulose phthalate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate, and any other pharmaceutically acceptable polymer that dissolves in phosphate buffer pH>5.5 or mixtures thereof.

According to a currently preferred embodiment said water soluble hydrophilic carrier is polyvinyl pyrrolidone.

According to one embodiment the subcoat further comprises at least one water insoluble particulate matter. According to various embodiments, said water insoluble particulate matter is selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon doxide and cross-linked polyacrylic acid

According to a currently preferred embodiment said water insoluble particulate matter is microcrystalline cellulose.

According to one embodiment the subcoat comprises povidone and microcrystalline cellulose.

In a particular embodiment, the subcoat surrounding the core comprises povidone and microcrystalline cellulose in a ratio 2:8 to 8:2, in a total amount of 0.5-5% (w/w) of core weight.

### Burst Core Release

An optional but preferred embodiment according to the present invention wherein the modified release core is preferably a burst release core. Without wishing to be limited by a single hypothesis, a preferred embodiment of the formulation according to the present invention preferably features a core which contains a swellable material, covered by a coating through which water enters the core. The swellable material in the core then swells and bursts the coating, after which the core more preferably disintegrates slowly or otherwise releases the active ingredient. Another optional but preferred embodiment relates to a fast disintegrating core.

Release of the active agent of the present formulation preferably occurs within about 2-6 hours of oral administration, with a slightly longer delay occurring with the enteric coated embodiment.

This optional embodiment of a formulation of the present invention therefore provides a delayed onset, rapid burst release formulation for delivery of statins in the lower GI tract preferentially to the colon or small intestine, which provides higher blood levels of statin or its metabolites in the bloodstream in comparison to a conventional immediate release formulation. The bioavailability is shown to be higher than that of a known reference product. The formulations according to the present invention should result in fewer side effects, greater safety, efficacy, and patient compliance.

This optional embodiment of a formulation of the present invention preferably includes a burst-controlling agent, such that release occurs rapidly, within a period of less than 8 hours following oral administration, with selective absorption of the active agent in the lower GI tract.

In one embodiment the delayed burst release formulation is based on a fast disintegrating core. The core can be based on either a swellable non hydrogel forming formulation or non swellable non hydrogel forming formulation, but in any case it is preferably a fast disintegrating formulation. The swellable or non swellable components thereto may optionally be water insoluble polymers as described herein, but alternatively may comprise one or more of osmotic pressure-creating agents such as water soluble salts (low molecular weight) and water soluble polymers such as polyvinyl pyrrolidone, carboxymethyl cellulose, hydroxyethyl cellulose, hydroxymethyl cellulose, hydroxypropyl cellulose.

Such a formulation can prevent release of the active ingredient in the stomach and even in the upper GI tract for a predetermined period of time, for example up to about 2 hours, more preferably up to about 3 to 4 hours, most preferably up to about 6 hours, after which the release can take place in a burst manner (fast release). The core according to such an embodiment may comprise the active ingredient, a disintegrant and a burst controlling agent which is preferably a water swellable non hydrogel forming polymer, in which the core is preferably formed as a compressed tablet. More preferably, the core is in the form of one of a tablet, pellets, microparticles, agglomerate, and capsule.

The core may comprise the active ingredient, a filler and a disintegrant, or alternatively the active ingredient and one or more disintegrants.

More preferably, the burst controlling agent comprises a water insoluble polymer. Most preferably, the water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

Preferably, the cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof.

Preferably, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

Also most preferably, the water insoluble polymer is calcium pectinate or microcrystalline cellulose.

Optionally and preferably, the disintegrant is selected from the group consisting of croscarmellose sodium, crospovidone (cross-linked polyvinyl pyrrolidone) sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, magnesium aluminum silicate and a combination thereof. More preferably, the disintegrating agent is croscarmellose sodium. Croscarmellose sodium is added to the core formulation in two distinct parts. The first part is added as an inter-granulate disintegrant, whose function is mainly to cause disaggregation of the granulate. The second part is added to the core apart from the granulate, where its function is to disintegrate the core.

The mechanism of disintegration is optionally based on swelling, wicking, and deformation of the disintegrants. Some commercial superdisintegrants for use in the present invention include, Ac-Di-Sol, Primojel, Explotab, and Crospovidone.

Preferably, the core further comprises at least one of an absorption enhancer, a binder, a hardness enhancing agent, and another excipient. More preferably, the binder is selected from the group consisting of Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Optionally, the core also includes a stabilizer. More preferably, the stabilizer comprises at least one of butyl hydroxyanisole, ascorbic acid and citric acid.

The core of the present invention optionally and preferably includes a wicking agent in addition to or as an alternative to a disintegrant. Wicking agents such as those materials already mentioned as disintegrants (e.g. microcrystalline cellulose) may be included if necessary to enhance the speed of water uptake. Other materials suitable for acting as wicking agents include, but are not limited to, colloidal silicon dioxide, kaolin, titanium dioxide, fumed silicon dioxide, alumina, niacinamide, sodium lauryl sulfate, low molecular weight polyvinyl pyrrolidone, m-pyrol, bentonite, magnesium aluminum silicate, polyester, polyethylene, mixtures thereof, and the like.

Alternatively or additionally, the core further comprises a filler. Preferably, the filler is selected from the group consisting of microcrystalline cellulose, starch, lactitol, lactose, a suitable inorganic calcium salt, sucrose, or a combination thereof. More preferably the filler is lactose monohydrate.

More preferably, the core further includes a chelating agent to increase chelation of trace quantities of metals thereby helping in preventing the loss of a statin such as Simvastatin by oxidation. Most preferably, the chelating agent is citric acid.

According to preferred embodiments of the present invention, the core further comprises a synergistic agent (sequestrate). Preferably, the sequestrate is selected from the group consisting of citric acid and ascorbic acid.

Without wishing to be limited by a single hypothesis, chelating agents and sequestrates may optionally be differentiated as follows. A chelating agent, such as (preferably) citric acid is intended to help in chelation of trace quantities of metals thereby assisting to prevent the loss of the active ingredient(s), such as a statin such as Simvastatin for example, by oxidation.

A sequestrate such as (preferably) ascorbic acid, optionally and preferably has several hydroxyl and/or carboxylic acid groups, which can provide a supply of hydrogen for regeneration of the inactivated Butyl hydroxyanisole (BHA) antioxidant free radical. A sequestrate therefore preferably acts as a supplier of hydrogen for rejuvenation of the primary antioxidant.

According to preferred embodiments of the present invention, the core further comprises an antioxidant. Preferably, the antioxidant is selected from the group consisting of 4,4 (2,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid.

More preferably, the core further comprises ascorbic acid, which has several hydroxyl and/or carboxylic acid groups, and is able to provide a supply of hydrogen for regeneration of the primary antioxidant, exerting a synergistic effect on the inactivated antioxidant free radical. Most preferably, the primary antioxidant is BHA. According to preferred embodiments of the present invention, the core further comprises a chelating agent. Preferably, the chelating agent is selected from the group consisting of Antioxidants, Dipotassium edentate, Disodium edentate, Edetate calcium disodium, Edetic acid, Fumaric acid, Malic acid, Maltol, Sodium edentate, Trisodium edetate. Also alternatively or additionally, the core further comprises a flow regulating agent. Preferably, the flow regulating agent includes at least one of colloidal silicon dioxide and aluminum silicate.

Most preferably, the flow regulating agent is colloidal silicon dioxide. Preferably, the core further comprises a lubricant. More preferably, the lubricant is selected from the group consisting of stearate salts; stearic acid, corola oil, glyceryl palmitostearate, hydrogenated vegetable oil, magnesium oxide, mineral oil, poloxamer, polyethylene glycole, polyvinyl alchol, sodium benzoate, talc, sodium stearyl fumarate, compritol (glycerol behenate), and sodium lauryl sulfate (SLS) or a combination thereof. Most preferably, the lubricant is magnesium stearate.

### Outer Coating

In a preferred embodiment, the outer coating comprising a water insoluble hydrophobic carrier and a water insoluble hydrophilic particulate matter, the water insoluble hydrophilic particulate matter allowing entry of liquid into said core.

In one embodiment, said water insoluble hydrophilic particulate matter forms channels in said outer coating upon contact with a liquid, whereby said channels absorb said liquid and cause said at least one burst controlling agent to burst said coating, thereby providing delayed burst release of said statin.

The water-insoluble hydrophobic carrier is preferably a water insoluble polymer. Examples of suitable hydrophobic carriers include but are not limited to dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, said polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes.

The water-insoluble, hydrophilic particulate matter in the outer coating is preferably a water insoluble but permeable polymer. Non-limiting examples of such polymers include a water insoluble cross-linked polysaccharide, a water insoluble cross-linked protein, a water insoluble cross-linked peptide, water insoluble cross-linked gelatin, water insoluble cross-linked hydrolyzed gelatin, water insoluble cross-linked collagen, water insoluble cross linked polyacrylic acid, water insoluble cross-linked cellulose derivatives, water insoluble cross-linked polyvinyl pyrrolidone, micro crystalline cellulose, insoluble starch, micro crystalline starch and a combination thereof. According to one currently preferred embodiment, the water insoluble particulate matter is micro crystalline cellulose. According to another currently preferred embodiment, the water-insoluble hydrophilic particulate matter comprises a mixture of Avicel (microcrystalline cellulose) and Ethocel.

The coating may also contain a pH dependent coating film (featuring a pH dependent polymer), preferably an enteric coating; a combination of at least one water soluble polymer and at least one water insoluble polymer; a combination of at least one swellable polymer and at least one water insoluble polymer; a combination of at least a water soluble pore forming agent and at least one water insoluble polymer; at least one swellable gel forming polymer; at least one erodible polymer; a combination of at least one pH dependent polymer and at least one water insoluble polymer; or a two-layer coating comprising a rupturable outer layer and swellable inner layer. These coatings are preferred embodiments of coatings for the present invention since, without wishing to be limited by a single hypothesis, they are structured so as to provide a delayed burst release in combination with a suitable core. These coatings are capable either of disintegration or of partial or complete loss of integrity, thereby supporting rapid release of material after disintegration of the core. Preferably, the core is a rapidly disintegrating core, and its rapid disintegration is supported by these coatings.

Optionally and preferably, the water insoluble polymer is hydrophobic and hence does not form a hydrogel.

According to this embodiment of the present invention, the pH dependent polymer of the outer coating is selected from the group consisting of a hydroxypropylmethyl cellulose phthalate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate. A suitable enteric coating can be from Eudragit® polymers series (available from Rohm Pharma) which are polymeric lacquer substances based on acrylates and/or methacrylates. Suitable polymers which are slightly permeable to water, and exhibit a pH-dependent permeability include, but are not limited to, Eudragit® L, and Eudragit® S. Eudragit® L is an anionic polymer synthesized from methacrylic acid and methacrylic acid methyl ester. It is insoluble in acids and pure water. It becomes soluble in neutral to weakly alkaline conditions. The permeability of Eudragit® L is pH dependent. Above pH 5.0, the polymer becomes increasingly permeable.

An illustrative, non-limiting example of such a formulation is as follows. The formulation optionally and preferably comprises a pH dependent film coat, the polymeric material comprises methacrylic acid co-polymers, ammonio methacrylate co-polymers, or a mixture thereof. Methacrylic acid co-polymers such as Eudragit® S and Eudragit® L (Rohm Pharma) are suitable for use in the delayed onset, modified, release formulations of the present invention, these polymers are gastro-resistant and entero-soluble polymers, providing a delay in onset of the release depending on the pH, the type of the polymer (Eudragit® L or Eudragit® S) and the thickness of the film coat.

The films of Methacrylic acid co-polymers such as Eudragit® S and Eudragit® L are insoluble in pure water and diluted acids. They dissolve at higher pH values, depending on their content of carboxylic acid. Eudragit® S and Eudragit® L can be used as single components in the coating of the formulation of the present invention or in combination in any ratio. By using a combination of the polymers, the polymeric material may exhibit a solubility at a pH between the pHs at which Eudragit® L and Eudragit® S are separately soluble.

Optionally, the outer coating further comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

In another embodiment according to the present invention the delayed onset, modified release formulation may comprise a fast disintegrating core formulation, as described above, and an outer coating, optionally comprising a combination of a water soluble polymer and/or a water swellable hydrophilic polymer and a water insoluble polymer. In this manner, where the film coating formulation features a combination of at least a water soluble polymer and at least a water insoluble polymer, it is possible to provide a delay time prior to the release of the active material, depending on the relative content (weight fraction) of the water soluble polymer in the outer coating, the thickness of the outer film coat, and the nature of the polymers present in the outer film coat. Without wishing to be limited by a single hypothesis, upon exposure of the formulation to the gastrointestinal fluids, the water soluble polymer starts to dissolve, leaving channels that allow penetration of the gastrointestinal fluids into the core, which may eventually lead to a relatively fast disintegration of the core and thus a burst release of the active material.

Another non-limiting, illustrative example of a suitable coating may be based on a core which can be formulated as described above for the previous embodiment, and an outer coating comprising a totally water soluble polymer and a water insoluble polymer. Suitable water-soluble polymers include, but are not limited to, polyvinyl alcohol, polyvinylpyrrolidone (PVP), methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof.

Suitable water insoluble polymers of the outer coating are selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly(butyl methacrylate), poly(isobutyl methacrylate), and poly(hexyl methacrylate), poly(isodecyl methacrylate), poly(lauryl methacrylate), poly(phenyl methacrylate), poly(methylacrylate), poly(isopropyl acrylate), poly(isobutyl acrylate) poly(octadecyl acrylate), poly(ethylene), poly(ethylene) low density, poly(ethylene) high density, poly(ethylene oxide), poly(ethyleneterephthalate), poly(vinyl isobutyl ether), poly(vinyl acetate), poly(vinyl chloride) and polyurethane, and/or mixtures thereof. More preferably, the water insoluble polymer is ethylcellulose.

An optional but preferred embodiment of such a coating comprises ethylcellulose (water insoluble polymer) and a copolymer of polyvinyl pyrrolidone and vinyl acetate (water soluble polymer). Preferably, the water insoluble polymer is present in an amount ranging from about 20% to about 95%, and the water soluble polymer is present in an amount ranging from about 5% to about 45% of the coating. More preferably, the coating further comprises a glidant. Most preferably, the glidant comprises Sieved Talc.

Optionally, the formulation may further comprise an enteric coating disposed on the outer coating.

Another non-limiting illustrative example of a coating may optionally feature an outer coating comprising a combination of a water swellable hydrophilic polymer and a water insoluble film-forming polymer. The swellable polymer may be a gel-forming polymer. This enables the swellable polymer participating in the outer film coat composition to be free of the requirement to fully dissolve. Since the swelling process of the swellable polymer in the outer film coat composition controls the diffusion process of the GI fluid through the film coat into the core, without wishing to be limited by a single hypothesis it is expected that it will be the only key factor for controlling the lag time. Another factor controlling the lag time is the thickness of the outer film coat.

Suitable swellable polymers typically interact with water and/or gastrointestinal fluids, which causes them to swell or expand to an equilibrium state. Acceptable polymers exhibit the ability to swell in water and/or gastrointestinal fluids, retaining a significant portion of such imbibed fluids within their polymeric structure. The polymers may swell or expand, usually exhibiting a 2- to 50-fold volume increase. The polymers can be non-cross-linked or cross-linked. The swellable hydrophilic polymer is responsible for introducing the gastrointestinal fluids into the core, leading to swelling of the core and eventually release of the active material, optionally through bursting of the core. The swellable polymers are hydrophilic polymers. Suitable polymers include, but are not limited to, poly(hydroxy alkyl methacrylate) having a molecular weight of from 30,000 to 5,000.000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity of hydroxylpropylmethyl cellulose and/or mixtures of any of the foregoing.

The outer film coat may also optionally include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40 or 50%, based on the weight of the dry polymer.

An optional but preferred embodiment of the above coating features a coating in which the swellable polymer comprises hydroxypropyl methyl cellulose (HPMC) and the water insoluble polymer comprises Ethyl cellulose. Preferably, the water insoluble polymer is present in an amount ranging from about 20% to about 95%, and the swellable polymer is present in an amount ranging from about 5% to about 45% of the coating.

More preferably, the coating further comprises a surfactant. Most preferably, the surfactant comprises sodium lauryl sulphate (SLS). More preferably, the coating further comprises a stiffening agent. Most preferably, the stiffening agent comprises cetyl alcohol. More preferably, the coating further comprises a glidant. Most preferably, the glidant comprises sieved talc.

Optionally, the formulation may comprise an enteric coating disposed on the outer coating.

In another embodiment, the outer film coat comprises one or more water-insoluble film-forming polymers and one or more water-soluble pore-forming compounds. Suitable water-soluble pore-forming compounds include, but are not limited to, saccharose, sodium chloride, potassium chloride, polyvinylpyrrolidone, and/or polyethyleneglycol, water soluble organic acids, sugars and sugar alcohol. The pore-forming compounds may be uniformly or randomly distributed throughout the water insoluble polymer. Typically, the pore-forming compounds comprise about 1 part to about 35 parts for each about 1 to about 10 parts of the water insoluble polymers. The amount and particle size of pore-forming agent in the film coat, and the thickness of the outer film coat are expected to be the main parameters controlling the lag time. Optionally, the formulation may comprise an enteric coating disposed on the outer coating.

In another embodiment a delayed onset, modified release formulation based on a dry compress coating process may be considered. Such a dosage form may optionally feature a rapidly disintegrating core coated with an erodible composition using a double compress tabletation. Such an erodible composition may optionally feature slow dissolving or slow disintegrating pharmaceutically acceptable excipients such as, but not limited to, water soluble polymers that solubilize slowly, swellable polymer or a composition comprising a water soluble polymer that solubilizes slowly with a disintegrant or a swellable polymer with disintegrant. Alternatively the coating process can be carried out using a conventional coating process such as spraying of an erodible or swellable polymer. Such a solution may optionally include additional excipients like a disintegrant and talc.

When an erodible polymer is used, the erosion rate of such a coating may determine the lag time, therefore, the type of polymer being used as erodible polymer, may be expected to control the erosion rate of the coating can determine the lag time. Another parameter that can control the lag time is the amount of erodible polymer constituting the thickness of the coating.

When a swellable polymer is used, the coating layer, which typically comprises a hydrophilic gelling polymer or swellable polymer, swells on contact with gastro-intestinal juices to form a continuous film surrounding the core. The coating layer must sufficiently protect the integrity of the core for the desired period of time, without regard to the pH of the medium to which it is subjected. Once the desired, pre-delivery time period has elapsed, the core should be capable of relatively fast disintegration so that the statin is released in a burst manner at the predetermined delivery time.

The polymeric coating layer may comprise any suitable hydrophilic gelling polymer known to those skilled in the art. For example, suitable hydrophilic gelling polymers include but are not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, carbopol, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, sodium alginate, polyaminoacids, polyalcohols, polyglycols; and the like; and mixtures thereof. The preferred swellable polymeric coating layer comprises carbopol. The more preferred swellable polymeric coating layer comprises hydroxypropylmethylcellulose.

Alternatively, the swellable polymeric coating layer comprises other substances which are capable of becoming freely permeable with exactly defined kinetics following hydration in aqueous fluids. Such substances include but are not limited to saccharose, sorbitol, mannaese, and jaluronic acid; and the like.

In addition to the foregoing, the swellable polymeric coating layer may also include additional excipients such as lubricants, flow promoting agents, plasticizers, antisticking agents, natural and synthetic flavorings and natural and synthetic colorants. Specific examples of additional excipients include polyethylene glycol, polyvinylpyrrolidone, talc, magnesium stearate, glyceryl behenate, stearic acid, and titanium dioxide.

The swellable polymeric coating layer may be applied to the core using conventional film (or spray) coating techniques, double press coating or by the method involving the alternate application of binder and powdered polymeric coating particles.

In certain embodiments, gums for use in the compression coating include, for example and without limitation, heteropolysaccharides such as xanthan gum(s), homopolysaccharides such as locust bean gum, galactans, mannans, vegetable gums such as alginates, gum karaya, pectin, agar, tragacanth, acacia, carrageenan, tragacanth, chitosan, agar, alginic acid, other polysaccharide gums (e.g. hydrocolloids), and mixtures of any of the foregoing. Further examples of specific gums which may be useful in the compression coatings of the invention include but are not limited to acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, hibiscus, hordeum, letuca; lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing, and the like.

In certain especially preferred embodiments, the compression coating comprises a heteropolysaccharide such as xanthan gum, a homopolysaccharide such as locust bean gum, or a mixture of one or more hetero- and one or more homopolysaccharide(s). Heterodisperse excipients, previously disclosed as a sustained release tablet matrix in U.S. Pat. No. 4,994,276, U.S. Pat. No. 5,128,143, and U.S. Pat. No. 5,135,757, may be utilized in the compression coatings of the present invention. For example, in certain embodiments of the present invention, a gelling agent of both hetero- and homo-polysaccharides which exhibit synergism, e.g., the combination of two or more polysaccharide gums producing a higher viscosity and faster hydration than that which would be expected by either of the gums alone, the resultant gel being faster-forming and more rigid, may be used in the compression coatings of the present invention.

The term “heteropolysaccharide” as used in the present invention is defined as a water-soluble polysaccharide containing two or more kinds of sugar units, the heteropolysaccharide having a branched or helical configuration, and having excellent water-wicking properties and immense thickening properties.

An especially preferred heteropolysaccharide is xanthan gum, which is a high molecular weight (>10⁶) heteropolysaccharide. Other preferred heteropolysaccharides include derivatives of xanthan gum, such as deacylated xanthan gum, the carboxymethyl ether, and the propylene glycol ester.

The homopolysaccharide materials used in the present invention that are capable of cross-linking with the heteropolysaccharide include the galactomannans, i.e., polysaccharides that are composed solely of mannose and galactose. A possible mechanism for the interaction between the galactomannan and the heteropolysaccharide involves the interaction between the helical regions of the heteropolysaccharide and the unsubstituted mannose regions of the galactomannan. Galactomannans that have higher proportions of unsubstituted mannose regions have been found to achieve more interaction with the heteropolysaccharide. Hence, locust bean gum, which has a higher ratio of mannose to galactose, is especially preferred as compared to other galactomannans, such as guar and hydroxypropyl guar.

An additional embodiment comprises a tablet system featuring a fast disintegrating core, which is not necessarily swellable, coated with two distinct layers of swelling and rupturable coating layers, preferably comprising a rupturable outer layer and swellable inner layer in the two-layer coating. The rapidly disintegrating core containing statin is preferably coated sequentially with an inner swelling layer preferably containing superdisintegrant and an outer rupturable layer preferably comprising a brittle polymer. The latter coating layer may optionally include at least one permeation-enhancing agent in order to promote the diffusion of water into the rupturable coating layer. The swelling coating layer is responsible for bursting the outer coating layer (rupturable). This takes place when the swelling layer comes into the contact with water, where an internal force is exerted as a result of the swelling of this layer.

Such a coating has unique properties in that it is able to burst (split) independently of the core. The swellable inner layer is composed of a polymer or a combination of polymers being able to swell when contacted by water. The resulting osmotic pressure created from swelling can exert force on the rupturable outer layer to cause it to lose its integrity and eventually to burst. The swelling layer may be composed of a disintegrant embedded in a water soluble film forming polymer. Non-limiting examples of the polymers which can be utilized in the swellable inner layer are hydroxypropylmethyl cellulose, high molecular weight of carboxymethyl cellulose, high molecular weight of hydroxypropyl cellulose, high molecular weight of hydroxyethyl cellulose, high molecular weight of hydroxymethyl cellulose, polyhydroxyethyl methacrylate, polyhydroxymethyl methacrylate, polyacrylic acid, carbopol, polycarbophil, gums, polysaccharides, modified polysaccharides, cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid. According to specific embodiments, the cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof. According to specific embodiments, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose. The swellable inner layer can be also based on combination of a water soluble polymer and a swellable water insoluble polymer particulate which is embedded into the water soluble polymer film matrix.

The rupturable outer layer is a water insoluble polymer which can be selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes. More preferably, the water insoluble polymer is ethylcellulose.

According to an optional but preferred embodiment of the present invention, there is provided a coating comprising an enteric coating. Preferably, the enteric coating comprises Hydroxypropylmethyl cellulose acetate succinate (HPMC AS).

More preferably, HPMC AS is present in an amount ranging from about 25% to about 90% of the enteric coating. Optionally and more preferably, the coating comprises a plasticizer. Most preferably, the plasticizer comprises triethyl citrate. Also optionally and more preferably, the coating comprises a surfactant. Most preferably, the surfactant comprises sodium lauryl sulfate.

In various particular embodiments, the outer coating comprises microcrystalline cellulose PH-102, ethyl cellulose and preferably cetyl alcohol. In a particular embodiment the outer coating comprises microcrystalline cellulose and ethyl cellulose in a ratio 1:9 to 7:3, cetyl alcohol in amount 5-15% from the ethyl cellulose weight. Preferably, the outer coating further contains 3-8% sodium lauryl sulfate (SLS). The outer coating constitutes 3-50% (w/w) of core.

According to optional but preferred embodiments of the present invention, the coating comprises a combination of at least a water soluble pore forming agent and at least one water insoluble polymer. Optionally and preferably, the pore-forming agent is selected from the group consisting of saccharose, sodium chloride, potassium chloride, polyvinylpyrrolidone, and/or polyethyleneglycol, water soluble organic acids, sugars and sugar alcohol. Optionally, the pore forming compound is distributed uniformly throughout said water insoluble polymer. Alternatively, the pore forming compound is distributed randomly throughout said water insoluble polymer. Optionally, the pore-forming compound comprises about 1 part to about 35 parts for each about 1 to about 10 parts of said water insoluble polymer.

According to optional but preferred embodiments of the present invention, the coating comprises an erodible polymer. Optionally and preferably the erodible composition comprises at least one of a slow dissolving and a slow disintegrating composition. Preferably, the erodible composition comprises at least one of a slowly water soluble polymer and a swellable polymer. Also preferably, the erodible composition further comprises a disintegrant.

According to optional but preferred embodiments of the present invention, the coating comprises at least one swellable gel-forming polymer. Preferably, the swellable gel-forming polymer is selected from the group consisting of cellulosic polymers; vinyl polymers; acrylic polymers and copolymers, methacrylic acid copolymers, ethyl acrylate-methyl methacrylate copolymers, natural and synthetic gums, gelatin, collagen, proteins, polysaccharides, pectin, pectic acid, alginic acid, sodium alginate, polyaminoacids, polyalcohols, polyglycols; and mixtures thereof.

More preferably, the cellulosic polymer is selected from the group consisting of methylcellulose, carboxymethylcellulose, hydroxypropylcellulose, hydroxypropylmethylcellulose, and hydroxyethylcellulose. Most preferably, the cellulosic polymer comprises hydroxymethylcellulose.

Optionally and preferably, the coating comprises a water insoluble polymer that is swellable, although alternatively it may be non swellable.

According to optional but preferred embodiments of the present invention, the coating further comprises at least one of a lubricant, a flow promoting agent, a plasticizer, an antisticking agent, natural and synthetic flavorings and natural and synthetic colorants.

Preferably, the lubricant further comprises at least one of polyethylene glycol, polyvinylpyrrolidone, talc, magnesium stearate, glyceryl behenate, stearic acid, and titanium dioxide.

According to optional but preferred embodiments of the present invention, the coating comprises a combination of at least one swellable polymer and at least one water insoluble polymer.

According to optional but preferred embodiments of the present invention, the coating comprises a combination of at least one pH dependent polymer and at least one water insoluble polymer.

### Therapeutic Uses

The formulations of the present invention are capable of providing a therapeutically effective amount of a statin, a pharmaceutically acceptable salt or ester thereof or an active form thereof to a subject, for an extended period of time after the burst release occurs. The formulations according to the present invention have increased efficacy and provide at least a similar, if not greater, pharmaceutical effect with the active ingredient, using a significantly decreased dosage amount as compared with conventional formulations known in the art regarding reduce of elevated total cholesterol, low density lipoprotein cholesterol, apolipoprotein B, triglycerides and increase of high density lipoprotein cholesterol. Preferably, the formulations of the present invention contain the statin in an amount that is up to about 60% as compared to an immediate release formulation, yet provides at least similar pharmaceutical efficacy. Thus, the novel formulations of the present invention are more effective than conventional statin formulations currently in use, and can be used for treating high cholesterol, ischemic heart disease and myocardial infarction, or any other disease or condition for which statins are indicated. The formulations of the present invention may even lead to new indications for the use of delayed burst release of simvastatin and can be used in new populations of patients in which the conventional statin formulations are not used for at present. The formulations of the present invention preferably comprise at least one statin in a decreased dosage amount of up to about 50%, or, in other embodiments, up to 60% as compared to an immediate release formulation of the statin, while providing a substantially equivalent effect of lowering of LDL as a full dosage of the immediate release formulation.

Thus in one aspect, the present invention relates to a method for providing a therapeutically effective amount of a statin, a pharmaceutically acceptable salt or ester thereof or an active form thereof to a subject, comprising orally administering to the subject a modified release formulation as described herein, featuring a slowly disintegrating core, wherein the formulation releases substantially no statin in vitro for at least about 2 hours to about 6 hours, preferably at least about 2 hours, more preferably at least about 3 hours, also more preferably at least about 4 hours, also more preferably at least about 5 hours and most preferably at least about 6 hours. In other embodiments, the formulation releases substantially no statin in vitro for at least about 1 hour, or, in other embodiments, for at least about 1.5 hours.

According to another embodiment of the present invention, there is provided a delayed onset modified release formulation for providing an increased blood concentration of a statin and/or active forms of the statin, relative to that resulting from the administration of an equivalent dose of the conventional immediate release formulations, comprising: a swellable, rapidly disintegrating core comprising at least one statin and at least one release controlling agent and an outer coating over the core, providing delayed release.

According to yet another embodiment of the present invention, such a delayed onset modified release formulation features an erodible film outer coating over the core, providing delayed release. Optionally the outer coating features a pH dependent film coating. Also optionally and alternatively the outer coating features a combination of a water soluble polymer and/or a water swellable hydrophilic polymer and a water insoluble polymer.

According to yet another embodiment of the present invention, there is provided a formulation featuring a burst release core with a coating selected from the group consisting of a pH dependent coating film, preferably an enteric coating; a combination of at least one water soluble polymer and at least one water insoluble polymer; a combination of at least one swellable polymer and at least one water insoluble polymer; a combination of at least a water soluble pore forming agent and at least one water insoluble polymer; at least one swellable gel forming polymer; at least one erodible polymer; a combination of at least one pH dependent polymer and at least one water insoluble polymer; or a two-layer coating comprising a rupturable outer layer and swellable inner layer, wherein the formulation releases substantially no statin in vitro for at least about 1 hour, preferably for at least about 90 minutes and more preferably for at least about 2 hours. Optionally and preferably, at least about 60% of the statin is released in vitro about one hour after the delayed burst release occurs.

According to other embodiments of the present invention, any of the above described formulations may optionally be used for reducing stress on the liver of the subject treated by at least one other drug involved in liver metabolism when administering a statin.

According to yet other embodiments of the present invention, any of the above described formulations may optionally be used for reducing liver side effects including increased level of transaminases when administering a statin.

According to yet other embodiments of the present invention, any of the above described formulations may optionally be used for reducing muscle pain and/or level of CPK when administering a statin.

According to yet other embodiments of the present invention, any of the above described formulations may optionally be used for reducing gastrointestinal effects comprising one or more of nausea, dyspepsia, flatulence or constipation when administering a statin.

According to yet other embodiments of the present invention, any of the above described formulations may optionally be used for providing release of a statin or a pharmaceutically acceptable salt or ester or active form thereof that is not affected by food intake.

According to still other embodiments of the present invention, any of the above described formulations may optionally be characterized in that the in vivo blood plasma concentration of the statin and/or a pharmaceutically acceptable salt and/or ester thereof is substantially zero for at least about one hour after oral administration and is controlled by the lag time, providing an increased blood concentration of a statin and/or active forms of said statin, relative to that resulting from the administration of an equivalent dose of the conventional immediate release formulations. Optionally and preferably, the in vivo blood plasma concentration is extended at least 24 hours.

According to still other embodiments of the present invention, any of the above described formulations may optionally be characterized in that the statin is released in the small intestine and/or lower gastrointestinal tract resulting in increased formation of intestinally active forms of the statin.

According to still other embodiments of the present invention, any of the above described formulations may optionally be characterized in that the statin is released in the small intestine and/or lower gastrointestinal tract resulting in an increased concentration of at least one active forms in the blood. Optionally the formulation comprises a decreased dosage of the statin and/or the pharmaceutically acceptable salt and/or ester thereof. Preferably, the core comprises a dose of statin of no more than about one-half of a dose as compared to a corresponding immediate release formulation, but wherein a level of at least one statin active form after administration of the formulation is at least about a level of the active metabolite after administration of the corresponding immediate release formulation.

In another aspect, the invention provides a method for providing a therapeutically effective amount of a statin, a pharmaceutically acceptable salt or ester thereof or an active form thereof to a subject, comprising orally administering to the subject a delayed burst release formulation comprising:
a. a core comprising at least one statin or a pharmaceutically acceptable salt or ester thereof, and at least one burst controlling agent, wherein the burst controlling agent is a water insoluble polymer;
b. a subcoat surrounding the core comprising at least one water soluble hydrophilic carrier; and
c. an outer coating over the core, the outer coating comprising a water insoluble hydrophobic carrier and a water insoluble hydrophilic particulate matter, the water insoluble hydrophilic particulate matter allowing entry of liquid into said core.

In another aspect, the invention provides a method for providing enhanced bioavailability of a statin, a pharmaceutically acceptable salt or ester thereof or an active form thereof to the circulation of a subject, as measured by the AUC compared to a substantially similar dose of an immediate release formulation of said statin, comprising orally administering to the subject a delayed burst release formulation comprising:
a. a core comprising at least one statin or a pharmaceutically acceptable salt or ester thereof, and at least one burst controlling agent, wherein the burst controlling agent is a water insoluble polymer;
b. a subcoat surrounding the core comprising at least one water soluble hydrophilic carrier; and
c. an outer coating over the core, the outer coating comprising a water insoluble hydrophobic carrier and a water insoluble hydrophilic particulate matter, the water insoluble hydrophilic particulate matter allowing entry of liquid into said core.

In another aspect, the invention provides a method of providing a delayed fast release of a statin, a pharmaceutically acceptable salt or ester thereof or an active form thereof in the gastrointestinal tract of a subject, comprising orally administering to the subject a formulation comprising:
a. a core comprising at least one statin or a pharmaceutically acceptable salt or ester thereof, and at least one burst controlling agent, wherein the burst controlling agent is a water insoluble polymer;
b. a subcoat surrounding the core comprising at least one water soluble hydrophilic carrier; and
c. an outer coating over the core, the outer coating comprising a water insoluble hydrophobic carrier and a water insoluble hydrophilic particulate matter, the water insoluble hydrophilic particulate matter allowing entry of liquid into said core.

In another aspect, the invention provides for the use of a delayed burst release formulation comprising:
a. a core comprising at least one statin or a pharmaceutically acceptable salt or ester thereof, and at least one burst controlling agent, wherein the burst controlling agent is a water insoluble polymer;
b. a subcoat surrounding the core comprising at least one water soluble hydrophilic carrier; and
c. an outer coating over the core, the outer coating comprising a water insoluble hydrophobic carrier and a water insoluble hydrophilic particulate matter, the water insoluble hydrophilic particulate matter allowing entry of liquid into said core;
   for the preparation of a medicament. In various embodiments, the medicament is useful for providing a therapeutically effective amount of a statin, a pharmaceutically acceptable salt or ester thereof or an active form thereof to a subject; for providing enhanced bioavailability of a statin, a pharmaceutically acceptable salt or ester thereof or an active form thereof to the circulation of a subject, as measured by the AUC compared to a substantially similar dose of an immediate release formulation of said statin; and/or for providing a delayed fast release of a statin, a pharmaceutically acceptable salt or ester thereof or an active form thereof in the gastrointestinal tract of a subject.

In another aspect, there is provided a delayed burst release formulation comprising:
a. a core comprising at least one statin or a pharmaceutically acceptable salt or ester thereof, and at least one burst controlling agent, wherein the burst controlling agent is a water insoluble polymer;
b. a subcoat surrounding the core comprising at least one water soluble hydrophilic carrier; and
c. an outer coating over the core, the outer coating comprising a water insoluble hydrophobic carrier and a water insoluble hydrophilic particulate matter, the water insoluble hydrophilic particulate matter allowing entry of liquid into said core;
   for providing a therapeutically effective amount of a statin, a pharmaceutically acceptable salt or ester thereof or an active form thereof to a subject; for providing enhanced bioavailability of a statin, a pharmaceutically acceptable salt or ester thereof or an active form thereof to the circulation of a subject, as measured by the AUC compared to a substantially similar dose of an immediate release formulation of said statin; and/or for providing a delayed fast release of a statin, a pharmaceutically acceptable salt or ester thereof or an active form thereof in the gastrointestinal tract of a subject.

The Examples given below are intended only as illustrations of various embodiments of the present invention, and are not intended to be limiting in any way.

### Examples

The following are Examples provided for comparison of the different core types and coating types of the present invention:

### Example 1

### Materials and Methods

### A. Core Types:

1—Simvastatin 8 mg, 2% Colloidal silicon dioxide, weight 250 mg;

2—Simvastatin 10 mg, 2% Colloidal silicon dioxide, weight 300 mg;

3—Simvastatin 16 mg, 2% Colloidal silicon dioxide, weight 300 mg;

4—Simvastatin 10 mg, 0.71% Colloidal silicon dioxide, weight 316 mg;

5—Simvastatin 10 mg, 1.5% Colloidal silicon dioxide, weight 300 mg;

6—Simvastatin 10 mg, 2% Colloidal silicon dioxide, weight 300 mg;

7—Simvastatin 10 mg, 1.5% Colloidal silicon dioxide, 1.33% Sodium lauryl sulphate (SLS), weight 300 mg;

8—Simvastatin 20 mg, 1.5% Colloidal silicon dioxide, in a geometrical relation of 2/1 with type 5 cores, weight 600 mg;

9—Simvastatin 20 mg, w/o Silicon dioxide, 10% Crospovidone, weight 300 mg;

10—Simvastatin 20 mg, 1.5% Colloidal silicon dioxide, weight 300 mg;

11—Simvastatin 20 mg, 2% Colloidal silicon dioxide, weight 300 mg;

### B. Coating Types:

A—TCDS coating (Microcrystalline cellulose PH 102-57.7%, Ethyl cellulose 20-38.5%, Cetyl alcohol—3.8%);

B—a coating comprised of i) a pre-coating (Povidone K 30-50%, Microcrystalline cellulose PH 101-50.0%), and ii) an outer TCDS coating (Microcrystalline cellulose PH 102-57.7%, Ethyl cellulose 20-38.5%, Cetyl alcohol—3.8%);

C—TCDS with 5% SLS coating (Microcrystalline cellulose PH 102-54.8%, Ethyl cellulose 20-36.5%, Cetyl alcohol—3.7%, Sodium lauryl sulphate—5%);

D—a coating comprised of i) a pre-coating (Povidone K 30-50%, Microcrystalline cellulose PH 101-50.0%), and ii) an outer TCDS with 5% SLS coating (Microcrystalline cellulose PH 102-54.8%, Ethyl cellulose 20-36.5%, Cetyl alcohol—3.7%, Sodium lauryl sulphate—5%);

E—a type D coating that has been thermally cured in a 60° C. oven for 16 hours.

### C. Tablet Core Preparation:

The composition and the mode of preparation of type 1 cores ( containing 8 mg simvastatin and 2% colloidal silicon dioxide) are presented in Table 1 and hereinbelow:

TABLE 1
The composition of preparation of type 1 cores

| Materials: | % tablet core | Weight (mg/tab) |
|---|---|---|
| Simvastatin | 3.20% | 8.00 |
| Microcrystalline cellulose PH 101 | 3.91% | 9.78 |
| Lactose monohydrate | 4.00% | 10.01 |
| Butylhydroxyanisole (BHA) | 0.01% | 0.03 |
| Citric acid | 0.40% | 1.00 |
| Ascorbic acid | 0.80% | 2.00 |
| Polyvinylpyrrolidone (Povidone K 30) | 0.62% | 1.55 |
| Cross-linked carboxymethylcellulose | 0.26% | 0.66 |
| sodium (Croscarmellose sodium) | | |
| Granulation solvent | P. Water, Isopropanol | |
| Colloidal silicon dioxide | 2.00% | 5.00 |
| Croscarmellose sodium. | 2.00% | 5.00 |
| Microcrystalline cellulose PH 102 | 82.15% | 205.40 |
| Magnesium stearate | 0.64% | 1.60 |
| Total Core | 100.00% | 250.0 |

The cores of the Simvastatin 8 mg tablets (sample core 1) were composed from granulates which included: simvastatin; lactose monohydrate (filler); microcrystalline cellulose PH 101 (filler); povidone K 30 (binder); the following stabilizers: 1) ascorbic acid, 2) citric acid, and 3) BHA; and crospovidone as a disintegrant. The granules were further mixed with other excipients, including: microcrystalline cellulose PH 102 as a filler, colloidal silicon dioxide as a glidant and a swelling controlling agent, croscarmellose sodium as a disintegrant and magnesium stearate as a lubricant.

The granulate was prepared by a wet granulation process using a Diosna high-shear granulator and dried in Glatt fluidized-bed machine. The granulate was milled through a 812 micron sieve. Next, the granulate was dry-blended with colloidal silicon dioxide and croscarmellose sodium for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

The composition and the mode of preparation of type 2 cores (containing 10 mg simvastatin and 2% colloidal silicon dioxide) are presented in Table 2 and hereinbelow:

TABLE 2
The composition of type 2 cores

| Materials: | % tablet core | Weight (mg/tab) |
|---|---|---|
| Simvastatin | 3.33% | 10.00 |
| Microcrystalline cellulose PH 101 | 7.00% | 21.00 |
| Lactose monohydrate | 9.00% | 27.00 |
| Butylhydroxyanisole | 0.04% | 0.12 |
| Citric acid | 1.25% | 3.75 |
| Ascorbic acid | 2.50% | 7.50 |
| Povidone K 30 | 0.73% | 2.20 |
| Croscarmellose sodium | 0.49% | 1.46 |
| Granulation solvent | P. Water, Isopropanol | |
| Colloidal silicon dioxide | 2.00% | 6.00 |
| Croscarmellose sodium. | 2.00% | 6.00 |
| Microcrystalline cellulose PH 102 | 71.06% | 213.20 |
| Magnesium stearate | 0.60% | 1.80 |
| Total Core | 100.00% | 300.0 |

The cores of the Simvastatin 10 mg tablets (sample core 2) were composed from granulates which included: simvastatin; lactose monohydrate (filler); microcrystalline cellulose PH 101 (filler); povidone K 30 (binder); the following stabilizers: 1) ascorbic acid, 2) citric acid, and 3) BHA; and crospovidone as a disintegrant. The granules were further mixed with other excipients, including: microcrystalline cellulose PH 102 as a filler, colloidal silicon dioxide as a glidant and a swelling controlling agent, croscarmellose sodium as a disintegrant and magnesium stearate as a lubricant.

The granulate was prepared by a wet granulation process using a Diosna high-shear granulator and dried in Glatt fluidized-bed machine. The granulate was milled through a 812 micron sieve. Next, the granulate was dry-blended with colloidal silicon dioxide and croscarmellose sodium for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

The composition and the mode of preparation of type 3 cores (each containing 16 mg simvastatin and 2% colloidal silicon dioxide) are presented in Table 3 and hereinbelow:

TABLE 3
The composition of type 3 cores

| | | |
|---|---|---|
| Materials: | % tablet core | Weight (mg/tab) |

| Simvastatin | 5.33% | 16.00 |
|---|---|---|
| Microcrystalline cellulose PH 101 | 6.52% | 19.55 |
| Lactose monohydrate | 6.67% | 20.00 |
| Butylhydroxyanisole | 0.02% | 0.06 |
| Citric acid | 0.67% | 2.00 |
| Ascorbic acid | 1.33% | 4.00 |
| Povidone K 30 | 1.03% | 3.10 |
| Croscarmellose sodium | 0.43% | 1.30 |
| Granulation solvent | P. Water, Isopropanol | |
| Colloidal silicon dioxide | 2.00% | 6.00 |
| Croscarmellose sodium. | 2.00% | 6.00 |
| Microcrystalline cellulose PH 102 | 73.36% | 220.10 |
| Magnesium stearate | 0.63% | 1.90 |
| Total Core | 100.00% | 316.0 |

The cores of the Simvastatin 16 mg tablets (sample core 3) were composed from granulates which included: simvastatin; lactose monohydrate (filler); microcrystalline cellulose PH 101 (filler); povidone K 30 (binder); the following stabilizers: 1) ascorbic acid, 2) citric acid, and 3) BHA; and crospovidone as disintegrant. The granules were further mixed with other excipients, including: microcrystalline cellulose PH 102 as a filler, colloidal silicon dioxide as a glidant and a swelling controlling agent, croscarmellose sodium as a disintegrant and magnesium stearate as a lubricant.

The granulate was prepared by a wet granulation process using a Diosna high-shear granulator and dried in Glatt fluidized-bed machine. The granulate was milled through a 812 micron sieve. Next, the granulate was dry-blended with colloidal silicon dioxide and croscarmellose sodium for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

The composition and the mode of preparation of type 4, 5 and 6 cores (containing 10 mg simvastatin and 0.71% -2% colloidal silicon dioxide), are presented in Table 4 and hereinbelow:

TABLE 4
The composition of type 4, 5 and 6 cores

| | Sample Core 4 | | Sample Core 5 | | Sample Core 6 | |
|---|---|---|---|---|---|---|
| | % tablet | Weight | % tablet | Weight | % tablet | Weight |
| Materials: | core | (mg/tab) | core | (mg/tab) | core | (mg/tab) |
| Simvastatin | 3.33% | 10.00 | 3.33% | 10.00 | 3.33% | 10.00 |
| Microcrystalline cellulose | 6.87% | 20.60 | 6.87% | 20.60 | 6.87% | 20.60 |
| PH 101 | | | | | | |
| Lactose monohydrate | 8.33% | 25.00 | 8.33% | 25.00 | 8.33% | 25.00 |
| Butylhydroxyanisole | 0.02% | 0.06 | 0.02% | 0.06 | 0.02% | 0.06 |
| Citric acid | 0.67% | 2.00 | 0.67% | 2.00 | 0.67% | 2.00 |
| Ascorbic acid | 1.33% | 4.00 | 1.33% | 4.00 | 1.33% | 4.00 |
| Povidone K 30 | 1.00% | 3.00 | 1.00% | 3.00 | 1.00% | 3.00 |
| Croscarmellose sodium | 0.43% | 1.30 | 0.43% | 1.30 | 0.43% | 1.30 |
| | P. Water, | | P. Water, | | P. Water, | |
| Granulation solvent | Isopropanol | | Isopropanol | | Isopropanol | |
| Colloidal silicon dioxide | 0.71% | 2.25 | 1.50% | 4.50 | 2.00% | 6.00 |
| Croscarmellose sodium. | 2.00% | 6.00 | 2.00% | 6.00 | 2.00% | 6.00 |
| Microcrystalline cellulose | 75.95% | 240.00 | 73.91% | 221.74 | 73.41% | 220.24 |
| PH 102 | | | | | | |
| Magnesium stearate | 0.60% | 1.80 | 0.60% | 1.80 | 0.60% | 1.80 |
| Total Core | 100.00% | 316.0 | 100.00% | 300.0 | 100.00% | 300.0 |

The cores of the Simvastatin 10 mg tablets (sample cores 4, 5 and 6) were each composed from a granulate which included: simvastatin; lactose monohydrate (filler); microcrystalline cellulose PH 101 (filler); povidone K 30 (binder); the following stabilizers: 1) ascorbic acid, 2) citric acid, and 3) BHA; and croscarmellose sodium as a disintegrant. The granules were further mixed with other excipients, including: microcrystalline cellulose PH 102 as a filler, colloidal silicon dioxide as a glidant and a swelling controlling agent, croscarmellose sodium as a disintegrant and magnesium stearate as a lubricant.

Each granulate was prepared by a wet granulation process using a V-Processor. Each granulate was milled through a 812 micron sieve. Next, the granulates were dry-blended with colloidal silicon dioxide and croscarmellose for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

The composition and the mode of preparation of type 7 cores (containing 10 mg simvastatin, 1.5% colloidal silicon dioxide and 1.33% sodium lauryl sulphate) are presented in Table 5 and hereinbelow:

TABLE 5
The composition of type 7 cores

| | | Weight |
|---|---|---|
| Materials: | % tablet core | (mg/tab) |
| Simvastatin | 3.33% | 10.00 |
| Microcrystalline cellulose PH | 6.87% | 20.60 |
| 101 | | |
| Lactose monohydrate | 7.00% | 21.00 |
| Butylhydroxyanisole | 0.02% | 0.06 |
| Citric acid | 0.67% | 2.00 |
| Ascorbic acid | 1.33% | 4.00 |
| Povidone K 30 | 1.00% | 3.00 |
| Croscarmellose sodium | 0.43% | 1.30 |
| Sodium lauryl sulphate | 1.33% | 4.00 |

| | |
|---|---|
| Granulation solvent | P. Water, Isopropanol |

| | | |
|---|---|---|
| Colloidal silicon dioxide | 1.50% | 4.50 |
| Croscarmellose sodium. | 2.00% | 6.00 |
| Microcrystalline cellulose PH | 73.91% | 221.74 |
| 102 | | |
| Magnesium stearate | 0.60% | 1.80 |
| Total Core | 100.00% | 300.0 |

The cores of the Simvastatin 10 mg tablets (sample core 7) were composed from granulates which included: simvastatin; lactose monohydrate (filler); microcrystalline cellulose PH 101 (filler); povidone K 30 (binder); sodium lauryl sulphate (solubilizer); the following stabilizers: 1) ascorbic acid, 2) citric acid, and 3) BHA; and croscarmellose sodium as a disintegrant. The granules were further mixed with other excipients, including: microcrystalline cellulose PH 102 as a filler, colloidal silicon dioxide as a glidant and a swelling controlling agent, croscarmellose sodium as a disintegrant and magnesium stearate as a lubricant.

The granulate was prepared by a wet granulation process using a V-Processor. The granulate was milled through a 812 micron sieve. Next, the granulate was dry-blended with colloidal silica and croscarmellose for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

The composition and the mode of preparation of type 8 cores (containing 20 mg simvastatin and 1.5% colloidal silicon dioxide, in a geometrical relation of 2/1 with type 5 cores) are presented in Table 6 and hereinbelow:

TABLE 6
The composition of type 8 cores

| | | Weight |
|---|---|---|
| Materials: | % tablet core | (mg/tab) |
| Simvastatin | 3.33% | 20.00 |
| Lactose monohydrate | 6.87% | 41.20 |
| Microcrystalline cellulose PH 101 | 8.33% | 50.00 |
| Butylhydroxyanisole | 0.02% | 0.12 |
| Citric acid | 0.67% | 4.00 |
| Ascorbic acid | 1.33% | 8.00 |
| Povidone K 30 | 1.00% | 6.00 |
| Croscarmellose sodium | 0.43% | 2.60 |

| | |
|---|---|
| Granulation solvent | P. Water, Isopropanol |

| | | |
|---|---|---|
| Colloidal silicon dioxide | 1.50% | 9.00 |
| Croscarmellose sodium. | 2.00% | 12.00 |
| Microcrystalline cellulose PH 102 | 75.95% | 443.48 |
| Magnesium stearate | 0.60% | 3.60 |
| Total Core | 100.00% | 600.0 |

The of the Simvastatin 20 mg tablets (sample core 8) were composed from the same type of granulate of Core type 5 which included: simvastatin; lactose monohydrate (filler); microcrystalline cellulose PH 101 (filler); povidone K 30 (binder); the following stabilizers: 1) ascorbic acid, 2) citric acid, and 3) BHA; and croscarmellose sodium as disintegrant. The granules were further mixed with other excipients, including: microcrystalline cellulose PH 102 as a filler, colloidal silicon dioxide as a glidant and a swelling controlling agent, croscarmellose sodium as a disintegrant and magnesium stearate as a lubricant. The amounts of the excipients are in a geometrical relation of 1:2 with the amounts of the excipients in type 5 cores.

The granulate was prepared by a wet granulation process using a V-Processor. The granulate was milled through a 812 micron sieve. Next, the granulate was dry-blended with colloidal silica and croscarmellose for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

Table 7: The composition and the mode of preparation of type 9 cores (containing 20 mg simvastatin and 10% crospovidone, without colloidal silicon dioxide) are presented in Table 7 and hereinbelow:

TABLE 7
The composition of type 9 cores:

| Materials: | % tablet core | Weight (mg/tab) |
|---|---|---|
| Simvastatin | 3.33% | 20.00 |
| Lactose monohydrate | 6.87% | 22.00 |
| Microcrystalline cellulose PH 101 | 8.33% | 20.00 |
| Butylhydroxyanisole | 0.02% | 0.12 |
| Citric acid | 0.67% | 3.75 |
| Ascorbic acid | 1.33% | 7.50 |
| Povidone K 30 | 1.00% | 2.20 |
| Croscarmellose sodium | 0.43% | 1.46 |
| Granulation solvent | P. Water, Isopropanol | |
| Colloidal silicon dioxide | | |
| Crospovidone | 10.00% | 30.00 |
| Microcrystalline cellulose PH 102 | 63.82% | 191.47 |
| Magnesium stearate | 0.50% | 1.50 |
| Total Core | 100.00% | 300.0 |

### Mode of Preparation:

The cores of the Simvastatin 20 mg tablets (sample core 9) were composed from granulates which included: simvastatin; lactose monohydrate (filler); microcrystalline cellulose PH 101 (filler); povidone K 30 (binder); the following stabilizers: 1) ascorbic acid, 2) citric acid, and 3) BHA; and crospovidone as a disintegrant. The granules were further mixed with other excipients, including: microcrystalline cellulose PH 102 as a filler, croscarmellose sodium as a disintegrant and magnesium stearate as a lubricant.

The granulate was prepared by a wet granulation process using a Diosna high-shear granulator and dried in Glatt fluidized-bed machine. The granulate was milled through a 812 micron sieve. Next, the granulate was dry-blended with crospovidone for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

The composition and the mode of preparation of type 10 and 11 cores (containing 20 mg simvastatin and 1.5%-2% colloidal silicon dioxide) are presented in Table 8 and hereinbelow:

TABLE 8
The composition of type 10 and 11 cores

| | Example Core 10 | | Example Core 11 | |
|---|---|---|---|---|
| | % tablet | Weight | % tablet | Weight |
| Materials: | core | (mg/tab) | core | (mg/tab) |
| Simvastatin | 6.67% | 20.00 | 6.67% | 20.00 |
| Lactose monohydrate | 6.33% | 22.00 | 6.33% | 19.00 |
| Microcrystalline | 5.07% | 20.00 | 5.07% | 15.20 |
| cellulose PH 101 | | | | |
| Butylhydroxyanisole | 0.02% | 0.12 | 0.02% | 0.06 |
| Citric acid | 0.83% | 3.75 | 0.83% | 2.50 |
| Ascorbic acid | 1.67% | 7.50 | 1.67% | 5.00 |
| Povidone K 30 | 1.00% | 2.20 | 1.00% | 3.00 |
| Croscarmellose sodium | 0.43% | 1.46 | 0.43% | 1.30 |
| Granulation solvent | P. Water, | | P. Water, | |
| | Isopropanol | | Isopropanol | |
| Colloidal silicon dioxide | 1.50% | 4.50 | 2.00% | 6.00 |
| Croscarmellose sodium. | 2.00% | 6.00 | 2.00% | 6.00 |
| Microcrystalline | 73.88% | 221.64 | 73.38% | 220.14 |
| cellulose PH 102 | | | | |
| Magnesium stearate | 0.60% | 1.80 | 0.60% | 1.80 |
| Total Core | 100.00% | 300.0 | 100.00% | 300.0 |

The cores of the Simvastatin 20 mg tablets (sample cores 10 and 11) were each composed from a granulate which included: simvastatin; lactose monohydrate (filler); microcrystalline cellulose PH 101 (filler); povidone K 30 (binder); the following stabilizers: 1) ascorbic acid, 2) citric acid, and 3) BHA; and croscarmellose sodium as a disintegrant. The granules were further mixed with other excipients, including: microcrystalline cellulose PH 102 as a filler, colloidal silicon dioxide as a glidant and a swelling controlling agent, croscarmellose sodium as a disintegrant and magnesium stearate as a lubricant.

Each granulate was prepared by a wet granulation process using a V-Processor. The granulate was milled through a 812 micron sieve. Next, the granulate was dry-blended with colloidal silica and croscarmellose for 5 min. The obtained mixture was blended with microcrystalline cellulose for 30 min. Finally magnesium stearate was passed through a mechanical sieve equipped with a 600 micron screen into the mixture and blended for 3 min. The latter process resulted in the tabletting mixture. The tabletting mixture was then compressed with a KILIAN tabletting press equipped with suitable capsule-shaped punches.

### D. Coating Processes:

The composition and the mode of preparation of type A coatings (containing a TCDS coating) are presented in Table 9 and hereinbelow:

TABLE 9
The composition of Type A Coating (TCDS coating)

| Materials: | % (w/w) of coating |
|---|---|
| TCDS Coating: | 100% |
| Microcrystalline cellulose PH 102 | 57.7% |
| Ethyl Cellulose 20 | 38.5% |
| Cetyl alcohol | 3.8% |

### Mode of Preparation:

Initially 0.5 kg of ethyl cellulose 20 was dissolved in 11.1 kg ethanol to obtain a clear solution (4.5% w/w), to which 0.05 kg cetyl alcohol was added and mixed with the mechanical stirrer to complete dissolution. 0.75 kg of microcrystalline cellulose PH 102 was added and stirred to obtain a homogeneous suspension. The resulting suspension was stirred throughout the whole coating process.

The type A coating process was performed in a perforated pan coater using a spraying pressure of 1.5-2.5 Bar at outlet air temperature 40±4° C. The coated tablets were dried in the coater at 42±4° C. for about 20 minutes.

The composition and the mode of preparation of type B coating (Pre-coating, then TCDS coating) are presented in Table 10 and hereinbelow:

TABLE 10
The composition of Type B Coating (Pre-coating, then TCDS coating)

| Materials: | % (w/w) of coating |
|---|---|
| Pre-coating: | 100% |
| Povidone K 30 | 50% |
| Microcrystalline cellulose PH 101 | 50% |
| TCDS Coating: | 100% |
| Microcrystalline cellulose PH 102 | 57.7% |
| Ethyl Cellulose 20 | 38.5% |
| Cetyl alcohol | 3.8% |

### Mode of Preparation:

For Pre-coating, 0.065 kg of povidone K 30 was dissolved in 0.65 kg isopropanol to obtain a clear solution (10% w/w), to which 0.065 kg of microcrystalline cellulose PH 101 was added and stirred to obtain a homogeneous suspension. The resulting suspension was stirred throughout the whole pre-coating process.

For TCDS coating, 0.5 kg of ethyl cellulose 20 was dissolved in 11.1 kg ethanol to obtain a clear solution (4.5% w/w), to which 0.05 kg cetyl alcohol was added and mixed with the mechanical stirrer to complete dissolution. 0.75 kg of microcrystalline cellulose PH 102 was added and stirred to obtain a homogeneous suspension. The resulting suspension was stirred throughout the whole coating process.

The type B coating process was performed in a perforated pan coater using a spraying pressure of 1.5-2.5 Bar at outlet air temperature 40±4° C. The coated tablets were dried in the coater at 42±4° C. for about 20 minutes.

The composition and the mode of preparation of type C coating (TCDS coating with 5% SLS) are presented in Table 11 and hereinbelow:

TABLE 11
The composition of Type C Coating (TCDS coating with 5% SLS)

| Materials: | % (w/w) of coating |
|---|---|
| TCDS Coating | 100% |
| Microcrystalline cellulose PH 102 | 54.8% |
| Ethyl Cellulose 20 | 36.5% |
| Sodium lauryl sulphate | 5.0% |
| Cetyl alcohol | 3.7% |

### Mode of Preparation:

For TCDS coating with SLS, 0.5 kg of ethyl cellulose 20 was dissolved in 11.1 kg ethanol to obtain a clear solution (4.5% w/w), to which 0.05 kg cetyl alcohol was added and mixed with the mechanical stirrer to complete dissolution. 0.068 kg of sodium lauryl sulphate was added to previous solution and stirred to obtain a homogeneous suspension and then 0.75 kg of microcrystalline cellulose PH 102 was added and stirred to obtain a homogeneous suspension. The resulting suspension was stirred throughout the whole coating process.

The type C coating process was performed in a perforated pan coater using a spraying pressure of 1.5-2.5 Bar at outlet air temperature 40±4° C. The coated tablets were dried in the coater at 42±4° C. for about 20 minutes.

The composition and the mode of preparation of type D coating (Pre-coating, then TCDS coating with 5% SLS) are presented in Table 12 and hereinbelow:

TABLE 12
The composition of Type D Coating (Pre-coating, then TCDS
coating with 5% SLS)

| Materials: | % (w/w) of coating |
|---|---|
| Pre-coating: | 100% |
| Povidone K 30 | 50% |
| Microcrystalline cellulose PH 101 | 50% |
| TCDS Coating | 100% |
| Microcrystalline cellulose PH 102 | 54.8% |
| Ethyl Cellulose 20 | 36.5% |
| Sodium lauryl sulphate | 5.0% |
| Cetyl alcohol | 3.7% |

### Mode of Preparation:

For Pre-coating, 0.065 kg of povidone K 30 was dissolved in 0.65 kg isopropanol to obtain a clear solution (10% w/w), to which 0.065 kg of microcrystalline cellulose PH 101 was added and stirred to obtain a homogeneous suspension. The resulting suspension was stirred throughout the whole pre-coating process.

For TCDS coating with SLS, 0.5 kg of ethyl cellulose 20 was dissolved in 11.1 kg ethanol to obtain a clear solution (4.5% w/w), to which 0.05 kg cetyl alcohol was added and mixed with the mechanical stirrer to complete dissolution. 0.068 kg of sodium lauryl sulphate was added to previous solution and stirred to obtain a homogeneous suspension and then 0.75 kg of microcrystalline cellulose PH 102 was added and stirred to obtain a homogeneous suspension. The resulting suspension was stirred throughout the whole coating process.

The type D coating process was performed in a perforated pan coater using a spraying pressure of 1.5-2.5 Bar at outlet air temperature 40±4° C. The coated tablets were dried in the coater at 42±4° C. for about 20 minutes.

The composition and the mode of preparation of type E coating (Thermally cured type D coating) are presented hereinbelow:

Materials and mode of preparation: the same with coating type D; Coated tablets were thermally cured in an oven at 60° C. for 16 hours.

### E. Methods of Sample Analysis:

Dissolution tests were performed in apparatus type 1 (baskets), at 37° C., 100 rpm, using as media 900 ml of a 0.1N HCL buffer for 1 hour, and then using USP buffer pH 7.0 with 0.5% sodium lauryl sulphate (SLS). The results were analyzed using an HPLC method.

For analyzing the amount of active material remaining in the coating after fast disintegration, the following procedure was employed:

20 tablets were dissected by length and transferred to a disintegration tester. After allowing the tablets to disintegrate for 10 minutes in water at a temperature 37° C., the coating films were assayed for the presence of simvastatin, and the percent of active material remaining in the coating was calculated.

### F. Formulations.

Formulation 1-A: Type 1 cores (containing 8 mg Simvastatin and 2% colloidal silicon dioxide, core weight 250 mg) coated with type A coating (TCDS, coating weight 40 mg/tablet).

Formulation 2-A: Type 2 cores (containing 10 mg Simvastatin and 2% colloidal silicon dioxide; core weight 300 mg) coated with type A coating (TCDS; coating weight 34 mg/tablet).

Formulation 3-A: Type 3 cores (containing 16 mg Simvastatin and 2% colloidal silicon dioxide; core weight 300 mg) coated with type A coating (TCDS; coating weight 40 mg/tablet).

Formulation 4-B: Type 4 cores (containing 10 mg Simvastatin and 0.7% colloidal silicon dioxide, core weight 316 mg) coated with type B coating (Pre-coating, then TCDS; Pre-coating weight 4 mg/tablet, TCDS coating weight 33 mg/tablet).

Formulation 4-C: Type 4 cores (containing 10 mg Simvastatin and 0.7% colloidal silicon dioxide; core weight 316 mg) coated with type C coating (TCDS with 5% SLS; coating weight 40 mg/tablet).

Formulation 5-A: Type 5 cores (containing 10 mg Simvastatin and 1.5% colloidal silicon dioxide; core weight 300 mg) coated with type A coating (TCDS; coating weight 37 mg/tablet).

Formulation 5-B: Type 5 cores (containing 10 mg Simvastatin and 1.5% colloidal silicon dioxide; core weight 300 mg) coated with type B coating (pre-coating, then TCDS; pre-coating weight 4 mg/tablet, TCDS coating weight 32 mg/tablet).

Formulation 5-C: Type 5 cores (containing 10 mg Simvastatin and 1.5% colloidal silicon dioxide; core weight 300 mg) coated with type C coating (TCDS with 5% SLS; coating weight 38 mg/tablet).

Formulation 5-D: Type 5 cores (containing 10 mg simvastatin and 1.5% colloidal silicon dioxide; core weight 300 mg) coated with type D coating (Pre-coating, then TCDS with 5% SLS; pre-coating weight 6 mg/tablet, TCDS coating weight 36 mg/tablet).

Formulation 5-D: Type 5 cores (containing 10 mg simvastatin and 1.5% colloidal silicon dioxide; core weight 300 mg) coated with type D coating (Pre-coating, then TCDS with 5% SLS; Pre-coating weight 6 mg/tablet, TCDS coating weight 36 mg/tablet).

Formulation 6-D: Type 6 cores (containing 10 mg Simvastatin and 2% colloidal silicon dioxide; core weight 300 mg) coated with type D coating (Pre-coating, then TCDS with 5% SLS; Pre-coating weight 3 mg/tablet, TCDS coating weight 50 mg/tablet).

Formulation 6-E: Type 6 cores (containing 10 mg simvastatin and 2% colloidal silicon dioxide; core weight 300 mg) coated with type E coating (Pre-coating, then TCDS with 5% SLS, then thermal curing at 60° C. for 16 hours; pre-coating weight 3 mg/tablet, TCDS coating weight 50 mg/tablet).

Formulation 7-A: Type 7 cores (containing 10 mg Simvastatin, 1.33% SLS and 1.5% colloidal silicon dioxide; core weight 300 mg) coated with type A coating (TCDS; coating weight 34 mg/tablet).

Formulation 7-B: Type 7 cores (containing 10 mg simvastatin, 1.33% SLS and 1.5% colloidal silicon dioxide; core weight 300 mg) coated with type B coating (Pre-coating, then TCDS; pre-coating weight 4 mg/tablet, TCDS coating weight 31 mg/tablet).

Formulation 7-C: Type 7 cores (containing 10 mg Simvastatin, 1.33% SLS and 1.5% colloidal silicon dioxide; core weight 300 mg) coated with type C coating (TCDS with 5% SLS; coating weight 36 mg/tablet).

Formulation 8-A: Type 8 cores (containing 20 mg Simvastatin and 1.5% colloidal silicon dioxide; core weight 600 mg) coated with type A coating (TCDS; coating weight 62 mg/tablet).

Formulation 8-D: Type 8 cores (containing 20 mg Simvastatin and 1.5% colloidal silicon dioxide; core weight 600 mg) coated with type D coating (Pre-coating, then TCDS with 5% SLS; pre-coating weight 12 mg/tablet, TCDS coating weight 68 mg/tablet).

Formulation 9-A: Type 9 cores (containing 20 mg Simvastatin, 10% crospovidone, without colloidal silicon dioxide; core weight 300 mg) coated with type A coating (TCDS; coating weight 31 mg/tablet).

Formulation 9-B: Type 9 cores (containing 20 mg simvastatin, 10% crospovidone, without colloidal silicon dioxide; core weight 300 mg) coated with type B coating (pre-coating, then TCDS; pre-coating weight 4 mg/tablet, TCDS coating weight 33 mg/tablet).

Formulation 10-D: Type 10 cores (containing 20 mg simvastatin and 1.5% colloidal silicon dioxide; core weight 300 mg) coated with type D coating (Pre-coating, then TCDS with 5% SLS; pre-coating weight 3 mg/tablet, TCDS coating weight 47 mg/tablet).

Formulation 11-D: Type 11 cores (containing 20 mg Simvastatin and 2% colloidal silicon dioxide; core weight 300 mg) coated with type D coating (Pre-coating, then TCDS with 5% SLS; pre-coating weight 3 mg/tablet, TCDS coating weight 50 mg/tablet).

Formulation 11-E: Type 11 cores (containing 20 mg Simvastatin and 2% colloidal silicon dioxide; core weight 300 mg) coated with type E coating (Pre-coating, then TCDS with 5% SLS, then thermal curing at 60° C. for 16 hours; pre-coating weight 3 mg/tablet, TCDS coating weight 50 mg/tablet).

### Example 2

### Residual Active Material in the TCDS Coat After Total Disintegration of the Tablet

The TCDS coating film is composed of a combination of a hydrophobic water-insoluble polymer in which water-insoluble but hydrophilic particles are embedded. The hydrophobic polymer, however, may trap a fraction of the active material existing at the interface between the TCDS coat and the surface of the core, and thus prevent the active material from being released even after total disintegration of the tablet occurs. This is particularly relevant for that group of active materials whose solubility in water or aqueous solutions is relatively low. The solubility of Simvastatin is relatively low; therefore, it can be entrapped in the hydrophobic water-insoluble part of the TCDS coat rather than being totally released. This may be more critical when the disintegration of the coated tablet takes place where the amount of water is relatively low, such as in the colon, and then the bioavailability and thus AUC will be negatively affected.

In order to prevent or reduce the amount of the active material retained in the TCDS film coat, the ability of a water soluble subcoat (precoating) to reduce or prevent the direct adherence of the active material at the interface to the TCDS film coat was examined.

Table 13 hereinbelow presents the amounts of residual simvastatin retained in the TCDS film coat (expressed by weight percent relative to the total dose) in pre-coated or non pre-coated tablets that underwent total disintegration. The tested formulations were prepared as described in Example 1 herein.

TABLE 13
Active material retention in the TCDS coating (after 10 minutes disintegration of
selected tablets)

| | | | | | | AM retention |
|---|---|---|---|---|---|---|
| | | | Retention | | | ratio |
| Formulation#, | Dose | | in coating | % of AM | Storage | with/without |
| dose, coating | (mg) | Coating type | (mg) | retention | time | pre-coat |
| 1-A, 8 mg TCDS | 8 | TCDS | 1.26 | 15.75% | 15 month, | |
| coat-CLINICAL | | | | | 25° C. | |
| BATCH | | | | | | |
| 2-A, 10 mg TCDS | 10 | TCDS | 1.45 | 14.50% | 12 | |
| coat CLINICAL | | | | | month, | |
| BATCH | | | | | 25° C. | |
| 3-A, 16 mg TCDS | 16 | TCDS | 1.00 | 6.25% | 24 | |
| coat-CLINICAL | | | | | month, | |
| BATCH | | | | | 25° C. | |
| 9-A, 20 mg TCDS | 20 | TCDS | 0.95 | 4.77% | time 0 | |
| coat | | | | | | |
| 9-B, 20 mg | 20 | TCDS + Precoat | 0.07 | 0.35% | time 0 | 7.2% |
| TCDS + Pre-coat | | | | | | |
| 5-A, 10 mg TCDS | 10 | TCDS | 1.39 | 13.87% | time 0 | |
| coat | | | | | | |
| 6-D, 10 mg | 10 | TCDS + 5% SLS + | 0.04 | 0.40% | time 0 | 2.8% |
| TCDS + 5% SLS + | | Precoat | | | | |
| Pre-coat | | | | | | |
| CLINICAL | | | | | | |
| BATCH | | | | | | |
| 11-D, 20 mg | 20 | TCDS + 5% SLS + | 0.08 | 0.40% | time 0 | 8.3% |
| TCDS + 5% SLS + | | Precoat | | | | |
| Pre-coat | | | | | | |
| CLINICAL | | | | | | |
| BATCH | | | | | | |

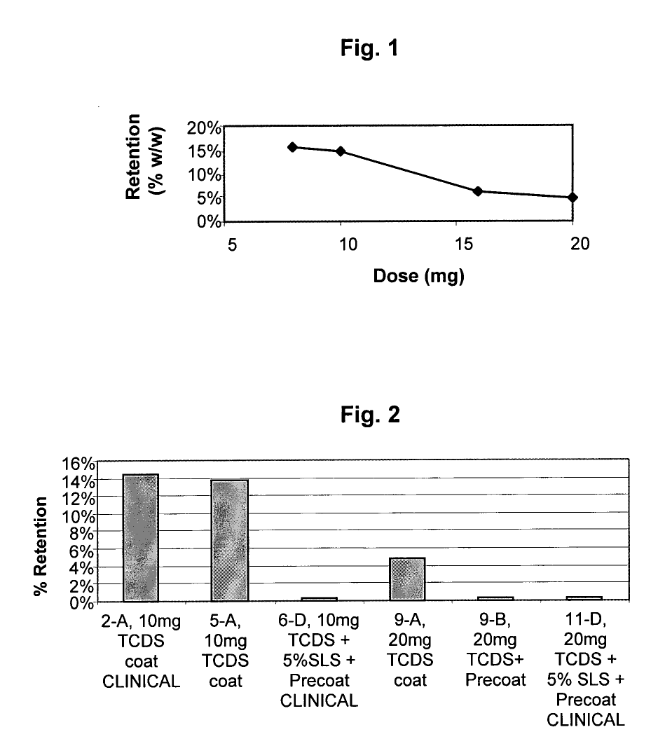

The retention of Simvastatin in a TCDS coating without a pre-coating as a function of the dosage is illustrated in FIG. 1. The retention of Simvastatin in a TCDS coating without a pre-coating with the different pre-coating procedures is illustrated in FIG. 2.

As shown in Table 13 and in FIG. 2, the pre-coating causes a significant reduction in the amount of residual active material retained in the TCDS coating after total disintegration of the core occurs. In contrast to the non pre-coated TCDS tablets, which exhibit a dose dependent retention (Examples 2-A and 5-A as compared to 9-A), the pre-coated TCDS tablets (Examples 6-D, 9-B, 11-D) were found to be dose independent.

### Example 3

### Dissolution Results

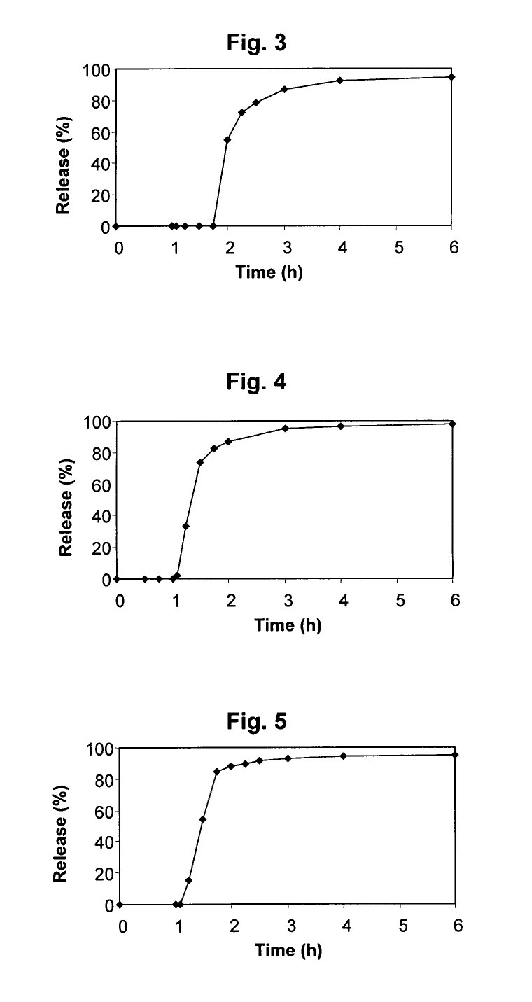

Tables 14-34 hereinbelow present the results of dissolution tests performed on test formulations 1-A, 2-A, 3-A, 4-B, 4-C, 5-A, 5-B, 5-C, 5-D, 6-D, 6-E, 7-A, 7-B, 7-C, 8-A, 8-D, 9-A, 9-B, 10-D, 11-D and 11-E, respectively, as described in Example 1 herein. FIGS. 3-23 are graphic representations of these results, wherein the accumulative release of Simvastatin (%) is presented as a finction of time (h).

Six tablet samples of each formulation, designated T1 to T6, were examined in each experiment. The mean values obtained for all six samples are designated “T-T6” in Tables 14 through 34.

TABLE 14
Dissolution test results - Simvastatin accumulative
release (%) - Formulation 1-A

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.0 | 0.0 | 0.0 | 0.0 | 0.0 | 0.0 | 0.0 |
| 1.5 | 0.0 | 0.0 | 0.0 | 0.0 | 0.0 | 0.0 | 0.0 |
| 1.75 | 0.0 | 0.0 | 0.0 | 0.0 | 0.0 | 0.0 | 0.0 |
| 2 | 53.3 | 49.5 | 66.2 | 29.7 | 64.9 | 67.4 | 55.2 |
| 2.25 | 65.3 | 72.7 | 75.3 | 65.2 | 75.7 | 78.2 | 72.1 |
| 2.5 | 70.9 | 79.8 | 82.5 | 71.1 | 81.8 | 84.1 | 78.4 |
| 3 | 79.4 | 87.2 | 89.8 | 78.7 | 89.4 | 95.7 | 86.7 |
| 4 | 93.1 | 92.2 | 91.7 | 87.0 | 93.2 | 97.6 | 92.5 |
| 6 | 93.7 | 93.8 | 91.9 | 92.9 | 94.3 | 99.9 | 94.4 |

FIG. 3 illustrates the accumulative release of Simvastatin (%) over time (h) for Formulation 1-A tablets. The amount of active material remained in the coating after fast disintegration was 15.8%.

TABLE 15
Dissolution test results - Simvastatin accumulative
release (%) - Formulation 2-A

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1 | 0.0 | 0.0 | 0.0 | 0.0 | 0.0 | 0.0 | 0.0 |
| 1.08 | 2.1 | 1.9 | 1.7 | 1.7 | 2.0 | 1.3 | 1.8 |
| 1.25 | 6.2 | 58.2 | 40.8 | 54.8 | 1.3 | 40.8 | 33.7 |
| 1.5 | 61.0 | 85.8 | 69.3 | 76.2 | 77.0 | 70.4 | 73.3 |
| 1.75 | 71.4 | 91.7 | 79.8 | 82.7 | 87.1 | 83.1 | 82.6 |
| 2 | 77.8 | 93.1 | 84.8 | 86.1 | 91.8 | 88.7 | 87.1 |
| 3 | 93.7 | 99.8 | 91.2 | 91.2 | 100.2 | 94.9 | 95.2 |
| 4 | 95.3 | 101.5 | 92.2 | 93.9 | 99.6 | 96.9 | 96.5 |
| 6 | 96.3 | 102.6 | 94.1 | 97.6 | 100.5 | 98.2 | 98.2 |

FIG. 4 illustrates the accumulative release of Simvastatin (%) over time for Formulation 2-A tablets. The amount of active material remained in the coating after fast disintegration was 14.5%.

TABLE 16
Dissolution test results - Simvastatin accumulative
release (%) - Formulation 3-A

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.0 | 54.6 | 0.0 | 0.0 | 0.0 | 35.6 | 15.0 |
| 1.5 | 74.1 | 80.8 | 83.4 | 0.0 | 0.0 | 86.8 | 54.2 |
| 1.75 | 82.7 | 84.7 | 88.4 | 84.8 | 77.5 | 89.2 | 84.5 |
| 2 | 88.5 | 87.2 | 89.1 | 86.6 | 86.4 | 90.9 | 88.1 |
| 2.25 | 89.7 | 88.6 | 90.2 | 87.3 | 89.2 | 91.4 | 89.4 |
| 2.5 | 91.6 | 89.4 | 91.7 | 90.2 | 92.3 | 93.2 | 91.4 |
| 3 | 93.8 | 90.0 | 92.7 | 92.7 | 96.2 | 92.8 | 93.0 |
| 4 | 95.7 | 93.3 | 95.1 | 92.3 | 97.3 | 92.4 | 94.3 |
| 6 | 97.1 | 92.5 | 95.4 | 94.0 | 98.2 | 92.5 | 94.9 |

FIG. 5 illustrates the accumulative release of Simvastatin (%) over time (h) for Formulation 3-A tablets. The amount of active material remained in the coating after fast disintegration was 6.3%.

TABLE 17
Dissolution test results - Simvastatin accumulative
release (%) - Formulation 4-B

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 0 | 0.0 | 0.0 | 0.0 | 0.0 | 0.0 | 0.0 | 0.0 |
| 1.08 | 0.0 | 0.0 | 0.0 | 0.0 | 0.0 | 0.0 | 0.0 |
| 1.25 | 68.5 | 2.0 | 0.0 | 0.0 | 0.0 | 60.5 | 21.8 |
| 1.5 | 78.4 | 54.4 | 39.0 | 1.2 | 61.1 | 82.2 | 52.7 |
| 1.75 | 82.7 | 72.0 | 67.6 | 38.9 | 75.0 | 90.7 | 71.1 |
| 2 | 85.2 | 84.4 | 73.6 | 48.6 | 81.8 | 93.5 | 77.9 |
| 2.25 | 87.5 | 89.3 | 78.4 | 53.5 | 85.9 | 94.5 | 81.5 |
| 2.5 | 88.7 | 90.4 | 80.5 | 56.9 | 88.2 | 95.0 | 83.3 |
| 3 | 91.8 | 92.6 | 83.1 | 61.2 | 92.0 | 94.7 | 85.9 |
| 4 | 92.7 | 93.6 | 84.5 | 69.6 | 92.4 | 94.5 | 87.9 |
| 6 | 94.0 | 94.3 | 87.2 | 78.5 | 92.6 | 94.6 | 90.2 |

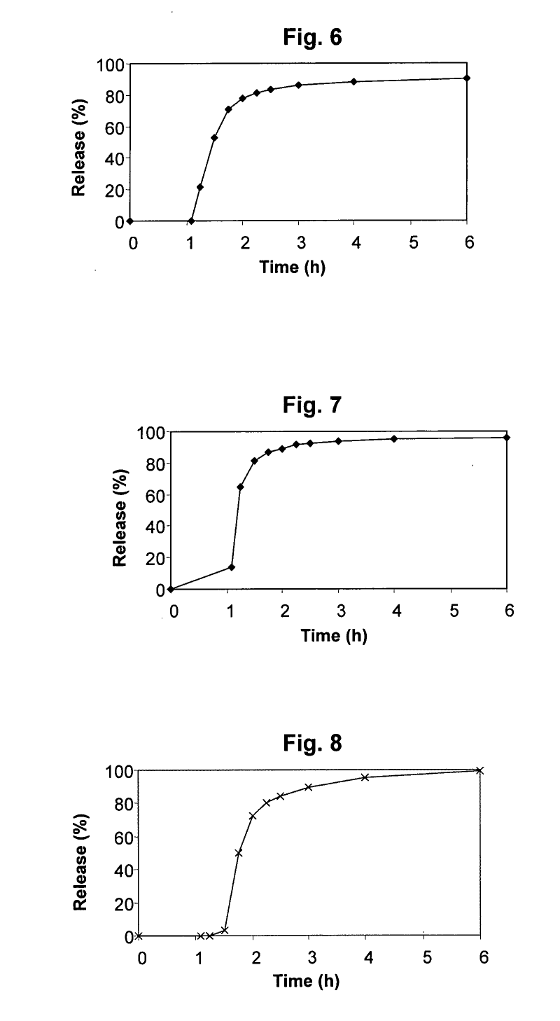

FIG. 6 illustrate the accumulative release of Simvastatin (%) over time (h) for Formulation 4-B tablets. The amount of active material remained in the coating after fast disintegration was 1.7%.

TABLE 18
Dissolution test results - Simvastatin accumulative
release (%) - Formulation 4C

| Hours | T1 | T2 | T3 | T4 | T5 | T6 | T1-T6 |
|---|---|---|---|---|---|---|---|
| 0 | 0.0 | 0.0 | 0.0 | 0.0 | 0.0 | 0.0 | 0.0 |
| 1.08 | 38.5 | 43.0 | 0.0 | 0.0 | 0.0 | 0.0 | 13.6 |
| 1.25 | 60.3 | 65.4 | 77.5 | 69.7 | 75.6 | 40.8 | 64.9 |
| 1.5 | 70.2 | 75.7 | 92.5 | 88.4 | 92.8 | 66.8 | 81.1 |
| 1.75 | 74.8 | 81.4 | 96.8 | 95.4 | 95.7 | 74.9 | 86.5 |
| 2 | 79.0 | 84.6 | 98.1 | 97.8 | 96.6 | 79.0 | 89.2 |
| 2.25 | 83.1 | 88.9 | 99.0 | 98.7 | 97.1 | 82.1 | 91.5 |
| 2.5 | 84.4 | 90.4 | 99.4 | 98.9 | 96.8 | 84.5 | 92.4 |
| 3 | 87.0 | 92.0 | 99.2 | 98.4 | 98.3 | 88.1 | 93.8 |
| 4 | 89.1 | 93.4 | 99.3 | 98.5 | 97.7 | 92.7 | 95.1 |
| 6 | 89.2 | 93.6 | 99.1 | 98.3 | 97.1 | 97.3 | 95.8 |

FIG. 7 illustrates the accumulative release of Simvastatin (%) over time (h) for Formulation 4C tablets. The amount of active material remained in the coating after fast disintegration was 4.2%.

TABLE 19
Dissolution test results - Simvastatin accumulative
release (%) - Formulation 5-A

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.0 | 0.0 | 0.0 | 0.0 | 0.0 | 0.0 | 0.0 |
| 1.5 | 0.0 | 0.0 | 0.0 | 0.0 | 0.0 | 18.7 | 3.1 |
| 1.75 | 52.0 | 0.0 | 54.5 | 65.0 | 54.1 | 75.4 | 50.2 |
| 2 | 68.6 | 48.7 | 77.0 | 74.5 | 81.9 | 85.2 | 72.7 |
| 2.25 | 76.1 | 63.8 | 82.4 | 79.4 | 92.0 | 88.3 | 80.3 |
| 2.5 | 81.0 | 71.0 | 85.5 | 82.1 | 95.6 | 91.4 | 84.4 |
| 3 | 86.6 | 81.7 | 87.7 | 86.2 | 99.4 | 96.2 | 89.7 |
| 4 | 92.8 | 91.3 | 94.4 | 91.7 | 101.6 | 98.6 | 95.1 |
| 6 | 97.8 | 98.4 | 98.0 | 97.7 | 102.6 | 100.4 | 99.1 |

FIG. 8 illustrates the accumulative release of Simvastatin (%) over time (h) for Formulation 5-A tablets. The amount of active material remained in the coating after fast disintegration was 13.9%.

TABLE 20
Dissolution test results - Simvastatin accumulative release (%) -
Formulation 5-B

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.0 | 0.0 | 0.0 | 0.0 | 0.0 | 0.0 | 0.0 |
| 1.5 | 45.8 | 40.1 | 28.2 | 64.0 | 45.3 | 81.7 | 50.8 |
| 1.75 | 56.6 | 65.9 | 79.8 | 83.1 | 57.3 | 96.5 | 73.2 |
| 2 | 65.3 | 78.8 | 87.7 | 88.7 | 65.5 | 101.0 | 81.2 |
| 2.25 | 71.3 | 87.2 | 91.7 | 91.9 | 74.9 | 101.3 | 86.4 |
| 2.5 | 74.0 | 94.5 | 93.9 | 93.0 | 80.0 | 102.8 | 89.7 |
| 3 | 78.2 | 97.2 | 96.3 | 95.7 | 93.1 | 103.6 | 94.0 |
| 4 | 86.6 | 97.7 | 98.8 | 96.1 | 102.3 | 103.7 | 97.5 |
| 6 | 94.1 | 99.0 | 101.0 | 96.6 | 102.3 | 103.3 | 99.4 |

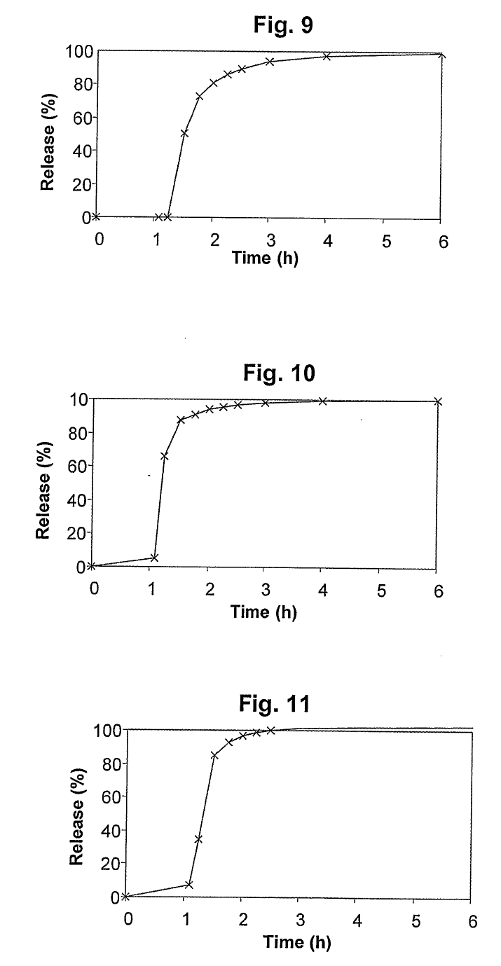

FIG. 9, illustrates the accumulative release of Simvastatin (%) over time (h) for Formulation 5-B tablets. The amount of active material remained in the coating after fast disintegration was 0.7%.

TABLE 21
Dissolution test results - Simvastatin accumulative release (%) -
Formulation 5-C

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 28.3 | 0.0 | 1.8 | 0.0 | 0.0 | 5.0 |
| 1.25 | 51.7 | 71.8 | 76.9 | 92.7 | 68.9 | 32.5 | 65.8 |
| 1.5 | 87.7 | 80.8 | 92.1 | 98.0 | 89.1 | 77.2 | 87.5 |
| 1.75 | 93.0 | 83.5 | 96.6 | 95.1 | 94.2 | 83.6 | 91.0 |
| 2 | 96.0 | 84.9 | 98.5 | 101.7 | 97.7 | 86.7 | 94.3 |
| 2.25 | 98.0 | 85.2 | 99.8 | 102.4 | 99.4 | 89.3 | 95.7 |
| 2.5 | 99.0 | 86.4 | 100.5 | 102.7 | 100.6 | 91.4 | 96.8 |
| 3 | 99.2 | 86.1 | 100.6 | 105.6 | 102.0 | 94.4 | 98.0 |
| 4 | 101.9 | 85.9 | 101.4 | 105.4 | 103.6 | 97.3 | 99.3 |
| 6 | 101.7 | 86.7 | 102.4 | 105.1 | 105.4 | 98.9 | 100.0 |

FIG. 10 illustrates the accumulative release of Simvastatin (%) over time (h) for Formulation 5-C tablets. The amount of active material remained in the coating after fast disintegration was 4.4%.

TABLE 22
Dissolution test results - Simvastatin accumulative release (%)
Formulation 5-D

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 16.3 | 0.0 | 22.7 | 0.0 | 3.4 | 0.0 | 7.1 |
| 1.25 | 42.1 | 0.0 | 60.4 | 50.5 | 52.8 | 2.4 | 34.7 |
| 1.5 | 90.0 | 73.4 | 86.4 | 78.3 | 95.0 | 88.7 | 85.3 |
| 1.75 | 97.2 | 81.8 | 96.6 | 83.9 | 98.8 | 99.0 | 92.9 |
| 2 | 100.0 | 87.6 | 98.9 | 87.1 | 104.8 | 102.0 | 96.7 |
| 2.25 | 100.1 | 91.2 | 99.6 | 89.9 | 104.8 | 106.4 | 98.7 |
| 2.5 | 101.0 | 94.5 | 101.9 | 91.2 | 104.8 | 106.4 | 100.0 |
| 3 | 101.1 | 100.6 | 101.7 | 92.3 | 104.3 | 106.8 | 101.1 |
| 4 | 101.0 | 102.5 | 102.0 | 95.9 | 103.0 | 107.5 | 102.0 |
| 6 | 101.9 | 102.3 | 100.8 | 99.2 | 104.6 | 107.4 | 102.7 |

FIG. 11 illustrates the accumulative release of Simvastatin (%) over time (h) for Formulation 5-D tablets. The amount of active material remained in the coating after fast disintegration was 0.9%.

TABLE 23
Dissolution test results - Simvastatin accumulative release (%) -
Formulation 6-D

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 0 | 0.0 | 0.0 | 0.0 | 0.0 | 0.0 | 0.0 | 0.0 |
| 1.08 | 0.0 | 0.0 | 0.0 | 0.0 | 0.0 | 0.0 | 0.0 |
| 1.25 | 0.0 | 0.0 | 0.0 | 0.0 | 0.0 | 0.0 | 0.0 |
| 1.5 | 0.0 | 0.0 | 0.0 | 0.0 | 0.0 | 0.0 | 0.0 |
| 1.75 | 69.4 | 72.2 | 48.3 | 71.5 | 68.8 | 56.7 | 64.5 |
| 2 | 82.4 | 88.0 | 84.9 | 83.0 | 83.7 | 75.5 | 82.9 |
| 2.25 | 87.8 | 94.5 | 89.4 | 86.6 | 88.9 | 81.8 | 88.2 |
| 2.5 | 90.3 | 98.6 | 91.7 | 88.1 | 91.7 | 85.7 | 91.0 |
| 3 | 95.2 | 102.2 | 93.9 | 91.1 | 95.7 | 89.2 | 94.5 |
| 4 | 98.9 | 103.4 | 96.5 | 96.0 | 99.1 | 94.0 | 98.0 |
| 6 | 102.0 | 104.7 | 101.2 | 100.6 | 101.0 | 98.2 | 101.3 |

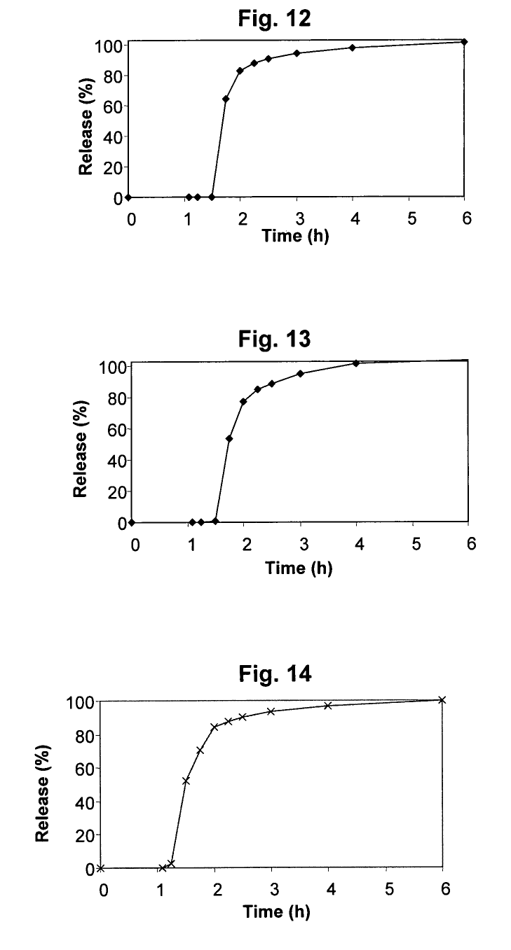

FIG. 12 illustrates the accumulative release of Simvastatin (%) over time (h) for Formulation 6-D tablets. The amount of active material remained in the coating after fast disintegration was 0.4%.

TABLE 24
Dissolution test results - Simvastatin accumulative release (%) -
Formulation 6-E

| Hours | T1 | T2 | T3 | T4 | T5 | T6 | T1-T6 |
|---|---|---|---|---|---|---|---|
| 0 | 0 | 0 | 0 | 0 | 0 | 0 | 0 |
| 1.08 | 0.0 | 0.0 | 0.0 | 0.0 | 0.0 | 0.0 | 0 |
| 1.25 | 0.0 | 0.0 | 0.0 | 0.0 | 0.0 | 0.0 | 0.0 |
| 1.5 | 0.0 | 0.0 | 0.0 | 0.0 | 0.0 | 5.7 | 0.9 |
| 1.75 | 32.0 | 55.1 | 45.1 | 57.4 | 66.1 | 64.8 | 53.4 |
| 2 | 70.7 | 75.2 | 72.5 | 80.7 | 89.9 | 76.5 | 77.6 |
| 2.25 | 79.0 | 81.7 | 83.3 | 87.4 | 96.2 | 81.2 | 84.8 |
| 2.5 | 84.8 | 85.8 | 89.7 | 91.8 | 96.1 | 83.6 | 88.6 |
| 3 | 90.7 | 91.5 | 99.0 | 97.7 | 100.8 | 91.8 | 95.2 |
| 4 | 100.3 | 100.2 | 103.8 | 101.9 | 104.7 | 98.1 | 101.5 |
| 6 | 103.9 | 104.3 | 104.0 | 104.4 | 106.0 | 100.2 | 103.8 |

FIG. 13, illustrates the accumulative release of Simvastatin (%) over time (h) for Formulation 6-E tablets. The amount of active material remained in coating after fast disintegration was 0.7%

TABLE 25
Dissolution test results - Simvastatin accumulative release (%) -
Formulation 7-A

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 6.9 | 7.3 | 0.0 | 0.0 | 0.0 | 2.4 | 2.8 |
| 1.5 | 77.7 | 90.9 | 3.4 | 54.3 | 83.2 | 3.3 | 52.1 |
| 1.75 | 85.2 | 97.8 | 68.0 | 73.2 | 93.6 | 5.0 | 70.5 |
| 2 | 89.4 | 100.1 | 75.3 | 78.9 | 96.9 | 64.1 | 84.1 |
| 2.25 | 95.9 | 100.9 | 78.1 | 81.9 | 97.6 | 71.4 | 87.6 |
| 2.5 | 99.8 | 101.3 | 81.3 | 83.8 | 99.5 | 74.4 | 90.0 |
| 3 | 103.1 | 102.2 | 85.1 | 88.0 | 102.1 | 79.2 | 93.3 |
| 4 | 105.1 | 104.0 | 91.0 | 92.6 | 101.7 | 84.7 | 96.5 |
| 6 | 108.0 | 106.4 | 97.1 | 95.7 | 104.0 | 90.7 | 100.3 |

FIG. 14, illustrates the accumulative release of Simvastatin (%) over time (h) for Formulation 7-A tablets. The amount of active material remained in the coating after fast disintegration was 15.7%.

TABLE 26
Dissolution test results - Simvastatin accumulative release (%) -
Formulation 7-B

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.0 | 0.0 | 38.7 | 0.0 | 0.0 | 0.0 | 6.5 |
| 1.5 | 45.3 | 49.7 | 52.3 | 20.0 | 0.9 | 69.4 | 39.6 |
| 1.75 | 61.5 | 63.0 | 60.1 | 50.1 | 37.9 | 81.8 | 59.1 |
| 2 | 69.0 | 67.6 | 64.4 | 56.1 | 56.8 | 86.3 | 66.7 |
| 2.25 | 72.4 | 70.2 | 66.6 | 59.7 | 63.9 | 87.6 | 70.1 |
| 2.5 | 74.8 | 72.4 | 68.5 | 62.5 | 68.2 | 87.8 | 72.4 |
| 3 | 78.0 | 75.6 | 72.1 | 66.5 | 74.7 | 88.9 | 76.0 |
| 4 | 82.5 | 80.2 | 78.0 | 74.8 | 81.3 | 90.1 | 81.1 |
| 6 | 85.7 | 88.8 | 81.8 | 82.1 | 85.6 | 90.7 | 85.8 |

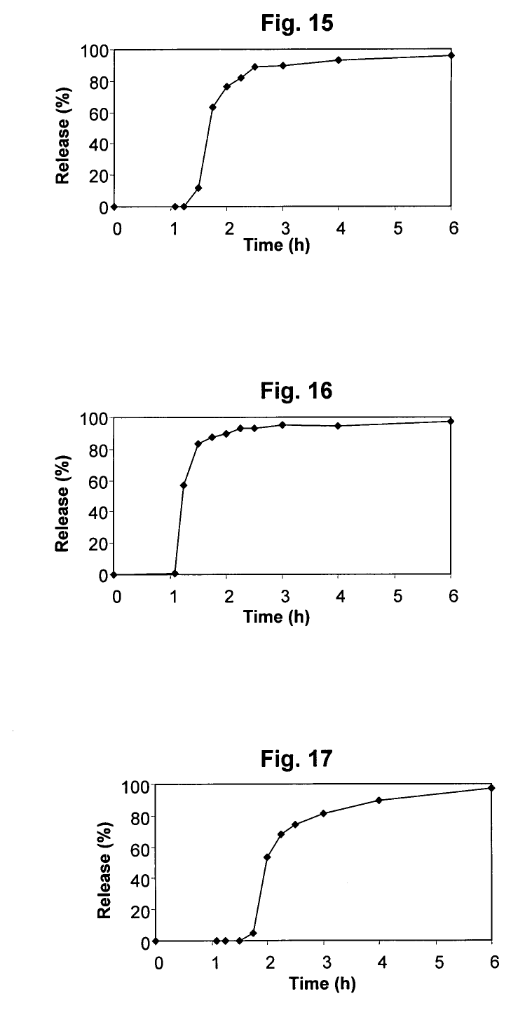

FIG. 15 illustrates the accumulative release of Simvastatin (%) over time (h) for Formulation 7-B tablets. The amount of Active material remained in the coating after fast disintegration was 2.1%.

TABLE 27
Dissolution test results - Simvastatin accumulative release (%) -
Formulation 7-C

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 3.9 | 0.0 | 0.6 |
| 1.25 | 68.3 | 58.1 | 65.4 | 1.1 | 83.1 | 64.5 | 56.8 |
| 1.5 | 82.0 | 77.8 | 81.8 | 88.5 | 90.2 | 80.3 | 83.4 |
| 1.75 | 86.0 | 85.8 | 86.2 | 95.0 | 92.3 | 80.8 | 87.7 |
| 2 | 86.5 | 90.5 | 88.5 | 95.9 | 92.0 | 84.0 | 89.6 |
| 2.25 | 92.9 | 93.8 | 94.5 | 95.7 | 95.9 | 87.3 | 93.3 |
| 2.5 | 92.1 | 94.3 | 92.9 | 97.5 | 95.3 | 88.1 | 93.4 |
| 3 | 93.1 | 97.6 | 97.1 | 97.9 | 96.5 | 89.2 | 95.2 |
| 4 | 94.3 | 93.5 | 96.7 | 96.8 | 96.6 | 88.5 | 94.4 |
| 6 | 94.7 | 100.8 | 98.6 | 99.1 | 96.5 | 92.0 | 97.0 |

FIG. 16 illustrates the accumulative release of Simvastatin (%) over time (h) for Formulation 7-C tablets. The amount of active material remained in the coating after fast disintegration was 1.7%.

TABLE 28
Dissolution test results - Simvastatin accumulative release (%) -
Formulation 8-A

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.0 | 0.0 | 0.0 | 0.0 | 0.0 | 0.0 | 0.0 |
| 1.5 | 0.0 | 0.0 | 0.0 | 0.0 | 0.0 | 0.0 | 0.0 |
| 1.75 | 9.1 | 0.0 | 19.9 | 0.7 | 0.0 | 0.0 | 4.9 |
| 2 | 65.1 | 53.4 | 54.9 | 41.9 | 63.3 | 40.7 | 53.2 |
| 2.25 | 73.5 | 70.6 | 65.2 | 59.4 | 83.8 | 55.2 | 68.0 |
| 2.5 | 78.6 | 75.9 | 70.9 | 68.9 | 90.7 | 61.7 | 74.4 |
| 3 | 84.9 | 81.6 | 78.4 | 79.1 | 95.4 | 69.0 | 81.4 |
| 4 | 91.0 | 89.0 | 88.3 | 89.0 | 100.3 | 79.1 | 89.4 |
| 6 | 100.3 | 95.8 | 99.4 | 95.5 | 102.0 | 88.9 | 97.0 |

FIG. 17 illustrates the accumulative release of Simvastatin (%) over time for Formulation 8-A tablets. The amount of active material remained in the coating after fast disintegration was 7.0%.

TABLE 29
Dissolution test results - Simvastatin accumulative release (%) -
Formulation 8-D

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1 | 0.0 | 0.0 | 0.0 | 0.0 | 0.0 | 0.0 | 0.0 |
| 1.08 | 30.5 | 2.2 | 0.7 | 3.6 | 0.0 | 19.8 | 9.5 |
| 1.25 | 68.6 | 4.3 | 41.3 | 32.7 | 51.4 | 42.5 | 40.1 |
| 1.5 | 79.9 | 57.0 | 66.8 | 77.4 | 66.5 | 55.4 | 67.2 |
| 1.75 | 78.8 | 64.9 | 75.8 | 81.0 | 71.2 | 76.1 | 74.6 |
| 2 | 84.5 | 68.5 | 79.6 | 82.7 | 74.6 | 86.4 | 79.4 |
| 2.25 | 85.3 | 71.1 | 81.4 | 81.2 | 76.4 | 90.9 | 81.1 |
| 2.5 | 85.6 | 72.9 | 82.5 | 85.3 | 76.7 | 91.6 | 82.5 |
| 3 | 86.5 | 75.6 | 84.9 | 88.0 | 78.5 | 92.9 | 84.4 |
| 4 | 87.7 | 79.9 | 88.3 | 94.7 | 80.1 | 93.8 | 87.4 |
| 6 | 86.8 | 84.5 | 93.2 | 97.1 | 82.3 | 94.0 | 89.7 |

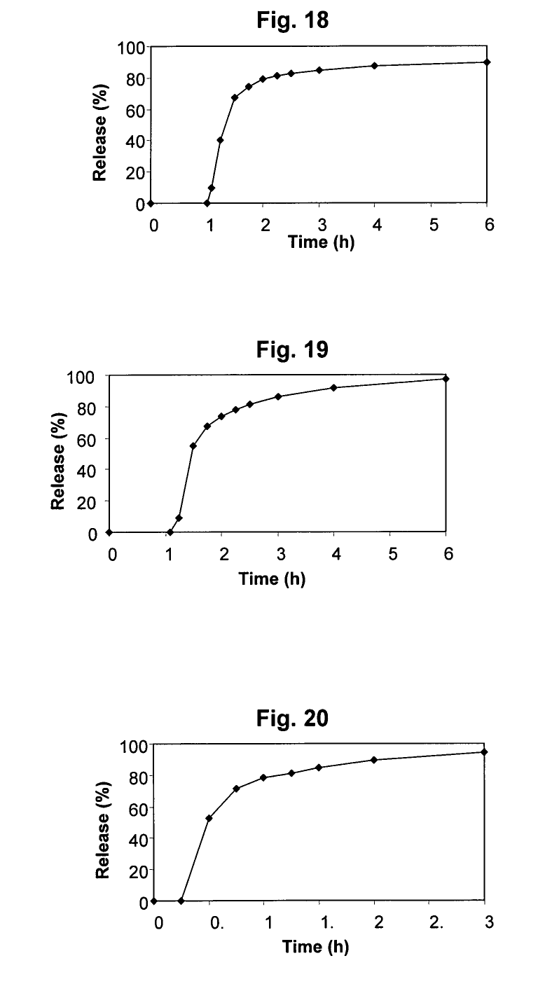

FIG. 18 illustrates the accumulative release of Simvastatin (%) over time (h) for Formulation 8-D tablets. The amount of active material remained in the coating after fast disintegration was 0.9%.

TABLE 30
Dissolution test results - Simvastatin accumulative release (%) -
Formulation 9-A

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 52.9 | 0.9 | 0.0 | 0.0 | 0.4 | 0.0 | 9.0 |
| 1.5 | 68.1 | 42.9 | 62.1 | 65.5 | 37.9 | 54.3 | 55.1 |
| 1.75 | 75.1 | 65.3 | 73.9 | 72.5 | 54.1 | 65.3 | 67.7 |
| 2 | 79.8 | 73.4 | 81.3 | 77.0 | 60.7 | 71.6 | 74.0 |
| 2.25 | 82.9 | 78.9 | 86.1 | 80.9 | 64.8 | 74.9 | 78.1 |
| 2.5 | 85.5 | 82.6 | 89.8 | 84.2 | 67.8 | 78.8 | 81.4 |
| 3 | 89.9 | 88.3 | 94.4 | 88.8 | 71.9 | 83.3 | 86.1 |
| 4 | 94.9 | 93.4 | 99.8 | 94.4 | 78.0 | 90.2 | 91.8 |
| 6 | 99.9 | 98.7 | 103.4 | 98.7 | 84.7 | 96.1 | 96.9 |

FIG. 19 illustrates the accumulative release of Simvastatin (%) over time (h) for Formulation 9-A tablets. The amount of active material remained in the coating after fast disintegration was 4.8%

TABLE 31
Dissolution test results - Simvastatin accumulative release (%) -
Formulation 9-B

| Hours | T1 | T2 | T3 | T4 | T1-T4 |
|---|---|---|---|---|---|
| 0 | 0 | 0 | 0 | 0 | 0.0 |
| 0.25 | 0.7 | 0.0 | 0.0 | 0.0 | 0.2 |
| 0.5 | 61.3 | 31.2 | 58.8 | 60.6 | 53.0 |
| 0.75 | 76.9 | 65.9 | 72.7 | 70.8 | 71.6 |
| 1 | 82.5 | 74.8 | 79.2 | 76.3 | 78.2 |
| 1.25 | 82.0 | 79.1 | 83.3 | 80.5 | 81.2 |
| 1.5 | 89.6 | 82.0 | 85.5 | 83.1 | 85.0 |
| 2 | 94.0 | 86.1 | 89.6 | 87.5 | 89.3 |
| 3 | 99.4 | 89.9 | 96.7 | 92.6 | 94.6 |

FIG. 20 illustrates the accumulative release of Simvastatin (%) over time (h) for Formulation 9-B tablets. The amount of active material remained in the coating after fast disintegration was 0.4%.

TABLE 32
Dissolution test results - Simvastatin accumulative release (%) -
Formulation 10-D

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.4 | 0.8 | 0.4 | 1.1 | 0.0 | 0.0 | 0.5 |
| 1.5 | 63.4 | 40.0 | 0.0 | 40.3 | 29.9 | 1.1 | 29.1 |
| 1.75 | 88.1 | 80.5 | 69.7 | 80.9 | 60.0 | 75.0 | 75.7 |
| 2 | 96.7 | 93.1 | 80.2 | 89.7 | 64.8 | 85.6 | 85.0 |
| 2.25 | 101.5 | 95.4 | 85.5 | 95.6 | 68.3 | 91.7 | 89.7 |
| 2.5 | 104.2 | 96.6 | 88.7 | 98.6 | 70.1 | 95.1 | 92.2 |
| 3 | 106.5 | 98.7 | 93.1 | 102.2 | 73.2 | 99.0 | 95.5 |
| 4 | 107.2 | 100.6 | 98.6 | 104.7 | 77.7 | 101.1 | 98.3 |
| 6 | 107.8 | 102.2 | 103.8 | 105.7 | 80.3 | 102.0 | 100.3 |

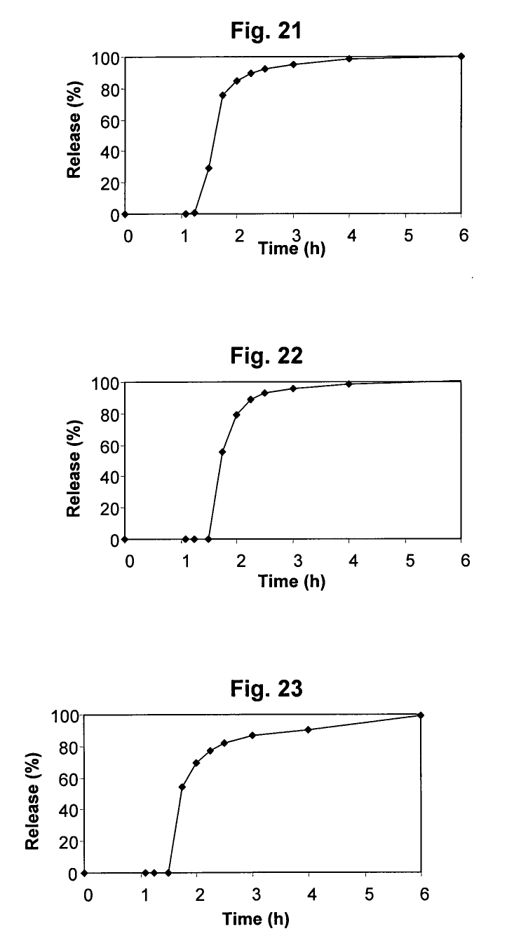

FIG. 21 illustrates the accumulative release of Simvastatin (%) over time (h) for Formulation 10-D tablets. The amount of active material remained in the coating after fast disintegration was 0.6%

TABLE 33
Dissolution test results - Simvastatin accumulative release (%) -
Formulation 11-D

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 0 | 0.0 | 0.0 | 0.0 | 0.0 | 0.0 | 0.0 | 0.0 |
| 1.08 | 0.3 | 0.0 | 0.0 | 0.4 | 0.3 | 0.3 | 0.2 |
| 1.25 | 0.0 | 0.0 | 0.0 | 0.0 | 0.0 | 0.0 | 0.0 |
| 1.5 | 0.0 | 0.0 | 0.0 | 0.0 | 0.0 | 0.0 | 0.0 |
| 1.75 | 60.8 | 43.8 | 82.0 | 57.7 | 56.3 | 34.5 | 55.9 |
| 2 | 79.8 | 76.4 | 94.1 | 81.7 | 72.7 | 71.8 | 79.4 |
| 2.25 | 84.7 | 89.3 | 97.7 | 92.0 | 78.9 | 90.4 | 88.8 |
| 2.5 | 87.2 | 93.1 | 100.3 | 96.3 | 83.1 | 96.7 | 92.8 |
| 3 | 90.4 | 95.9 | 102.2 | 99.8 | 87.2 | 100.1 | 95.9 |
| 4 | 94.1 | 98.1 | 104.2 | 102.2 | 92.2 | 101.8 | 98.8 |
| 6 | 98.3 | 100.2 | 104.8 | 102.7 | 95.8 | 102.7 | 100.8 |

FIG. 22, illustrates the accumulative release of Simvastatin (%) over time for Formulation 11-D tablets. The amount of active material remained in the coating after fast disintegration was 0.4%.

TABLE 34
Dissolution test results - Simvastatin accumulative release (%) -
Formulation 11-E

| Hours | T1 | T2 | T3 | T4 | T5 | T6 | T1-T6 |
|---|---|---|---|---|---|---|---|
| 0 | 0 | 0 | 0 | 0 | 0 | 0 | 0.0 |
| 1.08 | 0.0 | 0.0 | 0.0 | 0.0 | 0.0 | 0.0 | 0.0 |
| 1.25 | 0.0 | 0.0 | 0.0 | 0.0 | 0.0 | 0.0 | 0.0 |
| 1.5 | 0.0 | 0.0 | 0.0 | 0.0 | 0.0 | 0.0 | 0.0 |
| 1.75 | 46.3 | 63.5 | 68.3 | 48.6 | 54.9 | 43.0 | 54.1 |
| 2 | 58.8 | 76.8 | 81.7 | 73.5 | 72.2 | 54.8 | 69.6 |
| 2.25 | 65.1 | 82.8 | 90.9 | 83.4 | 76.6 | 62.0 | 76.8 |
| 2.5 | 71.7 | 86.0 | 94.9 | 90.2 | 80.5 | 67.5 | 81.8 |
| 3 | 80.0 | 90.0 | 96.6 | 95.3 | 83.7 | 74.8 | 86.7 |
| 4 | 84.4 | 93.5 | 96.3 | 98.4 | 87.3 | 80.4 | 90.0 |
| 6 | 98.6 | 102.1 | 96.8 | 103.3 | 97.4 | 98.3 | 99.4 |

FIG. 23 illustrates the accumulative release of Simvastatin (%) over time (h) for Formulation 11-E tablets. The amount of active material remained in the coating after fast disintegration was 0.8%.

As can be determined from Tables 14-34 and FIGS. 3-23, the presence of a pre-coat in the tablet coating markedly decreased the amount of active material retained within the coat.

### Example 4

### Bioequivalence Data

A bioequivalence study was performed in order to compare the relative bioavailability of three formulations of Simvastatin (two test products and one reference product). Two 10 mg Simvastatin Time Controlled Delivery System (TCDS) tablets, Dexcel Pharma Technologies (DPT) Ltd. (Israel) (Test Drug A) were compared to one 20 mg Simvastatin Time Controlled Delivery System (TCDS) tablet, Dexcel Pharma Technologies (DPT) Ltd. (Israel) (Test Drug B) and to the reference product Zocor 20 mg tablets, Merck Sharp & Dohme (MSD) UK Ltd. (UK), under fasting conditions. Both Test Drug Tablets contained a subcoat composed of PVP and microcrystalline cellulose and a TCDS coating which contained sodium lauryl sulphate. The tested formulations contained cores as presented in Table 35 and coating as presented in Table 36 hereinbelow:

TABLE 35
tablet cores

| | | Weight | | Weight |
|---|---|---|---|---|
| | | (mg/ | | (mg/ |
| | | tablet) - | | tablet) - |
| | | test | | test |
| Materials: | % tablet core | drug A | % tablet core | drug B |
| Simvastatin | 3.33% | 10.00 | 6.67% | 20.00 |
| Lactose monohydrate | 8.33% | 25.00 | 6.33% | 19.00 |
| Microcrystalline | 6.87% | 20.60 | 5.07% | 15.20 |
| cellulose PH 101 | | | | |
| Butylhydroxyanisole | 0.02% | 0.06 | 0.02% | 0.06 |
| Citric acid | 0.67% | 2.00 | 0.83% | 2.50 |
| Ascorbic acid | 1.33% | 4.00 | 1.67% | 5.00 |
| Povidone K 30 | 1.00% | 3.00 | 1.00% | 3.00 |
| Croscarmellose sodium | 0.43% | 1.30 | 0.43% | 1.30 |
| Granulation solvent | P. Water, | | P. Water, | |
| | Isopropanol | | Isopropanol | |
| Colloidal silicon dioxide | 2.00% | 6.00 | 2.00% | 6.00 |
| Croscarmellose sodium. | 2.00% | 6.00 | 2.00% | 6.00 |
| Microcrystalline | 73.41% | 220.24 | 73.38% | 220.14 |
| cellulose PH 102 | | | | |
| Magnesium stearate | 0.60% | 1.80 | 0.60% | 1.80 |
| Total Core | 100.00% | 300.0 | 100.00% | 300.0 |

tablet coating

| Materials: | % (w/w) of coating |
|---|---|
| Pre-coating: | 100% |
| Povidone K 30 | 50% |
| Microcrystalline cellulose PH 101 | 50% |
| TCDS Coating | 100% |
| Microcrystalline cellulose PH 102 | 54.8% |
| Ethyl Cellulose 20 | 36.5% |
| Sodium lauryl sulphate | 5.0% |
| Cetyl alcohol | 3.7% |

The study was designed as a randomized, three-way crossover study in healthy volunteers with a wash-out period of one week. Twelve healthy, male volunteers were planned for and concluded the study.

At each period, one or two tablets of either formulation was administered to fasting volunteers and blood samples were collected according to the following schedule: pre-dose, and 0.33, 0.67, 1, 1.5, 2, 2.5, 3, 3.5, 4, 4.5, 5, 6, 9, 12, 16, 24 and 36 hours post dosing.

The plasma concentrations of Simvastatin and of its main metabolite, β-hydroxyacid-simvastatin (SHA) were determined using the LC/MS/MS analytical method. The limit of quantification (LOQ), defined as the lowest concentration determined with accuracy and run-to-run precision lower than 20% was 0.100 ng/ml for Simvastatin and β-hydroxyacid-simvastatin (SHA).

All values below the limit of quantification (BLQ) were set to zero for pharmacokinetic and statistical computations.

A concentration-time curve was constructed for each volunteer for each period and for each analyte. The main pharmacokinetic parameter area under the curve (AUC) was computed from the plasma concentration-time curve using the trapezoidal method. The maximal concentration (Cmax), the time of its occurrence (Tmax) and the occurrence time of the first concentration above the Limit of Quantitation (Lag-time) were observed directly from the curves.

The 90% Confidence Interval, which is equivalent to that based on the two one-sided tests at a nominal significance level of 5% each, has become the standard in bioequivalence assessment. Thus, the 90% parametric (ANOVA) Confidence Intervals have been computed for ratios whenever possible.

The results are presented in Tables 37 (pharmacokinetic parameters obtained for simvastatin) and 38 (pharmacokinetic parameters obtained for SHA).

TABLE 37
Pharmacokinetic Parameters - Simvastatin

| | AUC₍₀₋₃₆₎ | Cmax | Tmax | Lag-time |
|---|---|---|---|---|
| | (ng × hour/ml) | (ng/ml) | (hours) | (hours) |
| TEST DRUG A: | 16.74 ± 8.14 | 1.72 ± 0.88 | 5.08 ± 2.58 | 2.29 ± 0.26 |
| Simvastatin 10 mg TCDS | (6.84; 33.22) | (0.70; 3.11) | (2.00; 12.00) | (2.00; 2.50) |
| Tablets | | | | |
| (Two Tablets) | | | | |
| B.N. 020206A (DPT) | | | | |
| TEST DRUG B: | 15.33 ± 8.24 | 1.79 ± 0.96 | 7.38 ± 4.34 | 2.29 ± 0.26 |
| Simvastatin 20 mg TCDS | (8.10; 35.57) | (0.62; 3.44) | (3.50; 16.00) | (2.00; 2.50) |
| Tablets | | | | |
| (One Tablet) | | | | |
| B.N. 290106B (DPT) | | | | |
| REFERENCE: | 8.12 ± 4.29 | 2.63 ± 1.89 | 1.25 ± 0.73 | 0.47 ± 0.18 |
| ZOCOR 20 mg Tablets | (1.80; 15.70) | (0.85; 7.73) | (0.67; 3.00) | (0.33; 0.67) |
| (One Tablet) | | | | |
| B.N. 254843 (MSD) | | | | |
| RATIO* | 2.22 | 0.69 | | |
| TEST DRUG A vs. | (1.73; 2.83) | (0.47; 1.02) | | |
| REFERENCE | | | | |
| 90% ANOVA C.I. | | | | |
| RATIO* | 2.00 | 0.71 | | |
| TEST DRUG B vs. | (1.44; 2.77) | (0.53; 0.95) | | |
| REFERENCE | | | | |
| 90% ANOVA C.I. | | | | |
| RATIO** | 1.11 | 0.98 | | |
| TEST DRUG A vs. TEST | (0.92; 1.34) | (0.70; 1.39) | | |
| DRUG B | | | | |
| 90% ANOVA C.I. | | | | |
| DIFFERENCE | | | 3.83 | 1.82 |
| ESTIMATE*** | | | (1.33; 11.33) | (1.33; 2.17) |
| TEST DRUG A vs. | | | | |
| REFERENCE | | | | |
| (range) | | | | |
| DIFFERENCE | | | 6.12 | 1.82 |
| ESTIMATE*** | | | (2.00; 14.50) | (1.33; 2.17) |
| TEST DRUG B vs. | | | | |
| REFERENCE | | | | |
| (range) | | | | |
| DIFFERENCE | | | −2.29 | 0.00 |
| ESTIMATE*** | | | (−10.00; 1.00) | (−0.50; 0.50) |
| TEST DRUG A vs. TEST | | | | |
| DRUG B | | | | |
| (range) | | | | |

The presented values for all pharmacokinetic parameters are mean ± SD and (range).
*The presented ratios are the geometric means of the ratios between the Test and Reference parameter, parametric estimators and parametric confidence intervals based on the linear model with logarithmic transformation (multiplicative model) are brought.
**The presented ratios are the geometric means of the ratios between Test drug A and Test drug B parameter, parametric estimators and parametric confidence intervals based on the linear model with logarithmic transformation (multiplicative model) are brought.
***The presented differences are the mean results and the range of Tmax.

TABLE 38
Pharmacokinetic Parameters - SHA

| | AUC₍₀₋₃₆₎ | | | |
|---|---|---|---|---|
| | (ng × | Cmax | Tmax | Lag-time |
| | hour/ml) | (ng/ml) | (hours) | (hours) |
| TEST DRUG A: | 11.40 ± 7.38 | 0.98 ± 0.68 | 8.92 ± 5.85 | 3.29 ± 0.78 |
| Simvastatin 10 mg TCDS | (3.07; 30.04) | (0.38; 2.57) | (4.50; 24.00) | (2.50; 4.50) |
| Tablets | | | | |
| (Two Tablets) | | | | |
| B.N. 020206A (DPT) | | | | |
| TEST DRUG B: | 9.78 ± 7.63 | 0.85 ± 0.68 | 8.08 ± 4.57 | 3.29 ± 0.94 |
| Simvastatin 20 mg TCDS | (1.65; 30.99) | (0.26; 2.75) | (4.50; 16.00) | (2.50; 5.00) |
| Tablets | | | | |
| (One Tablet) | | | | |
| B.N. 290106B (DPT) | | | | |
| REFERENCE: | 7.23 ± 5.07 | 0.82 ± 0.55 | 4.71 ± 0.58 | 0.81 ± 0.38 |
| ZOCOR 20 mg Tablets | (1.17; 20.42) | (0.26; 2.31) | (3.50; 6.00) | (0.33; 1.50) |
| (One Tablet) | | | | |
| B.N. 254843 (MSD) | | | | |
| RATIO* | 1.68 | 1.19 | | |
| TEST DRUG A vs. | (1.39; 2.04) | (0.94; 1.50) | | |
| REFERENCE | | | | |
| 90% ANOVA C.I. | | | | |
| RATIO* | 1.33 | 0.99 | | |
| TEST DRUG B vs. | (1.00; 1.76) | (0.77; 1.27) | | |
| REFERENCE | | | | |
| 90% ANOVA C.I. | | | | |
| RATIO** | 1.27 | 1.20 | | |
| TEST DRUG A vs. TEST | (0.98; 1.65) | (0.91; 1.59) | | |
| DRUG B | | | | |
| 90% ANOVA C.I. | | | | |
| DIFFERENCE | | | 4.21 | 2.49 |
| ESTIMATE*** | | | (−1.50; 19.50) | (1.50; 4.17) |
| TEST DRUG A vs. | | | | |
| REFERENCE | | | | |
| (range) | | | | |
| DIFFERENCE | | | 3.38 | 2.49 |
| ESTIMATE*** | | | (−0.50; 11.50) | (1.00; 4.33) |
| TEST DRUG B vs. | | | | |
| REFERENCE | | | | |
| (range) | | | | |
| DIFFERENCE | | | 0.83 | 0.00 |
| ESTIMATE*** | | | (−10.00; 19.50) | (−1.50; 1.50) |
| TEST DRUG A vs. TEST | | | | |
| DRUG B | | | | |
| (range) | | | | |

The presented values for all pharmacokinetic parameters are mean ± SD and (range).
*The presented ratios are the geometric means of the ratios between the test and Reference parameter, parametric estimators and parametric confidence intervals based on the linear model with logarithmic transformation (multiplicative model) are brought.
**The presented ratios are the geometric means of the ratios between Test drug A and Test drug B parameter, parametric estimators and parametric confidence intervals based on the linear model with logarithmic transformation (multiplicative model) are brought.
***The presented differences are the mean results and the range of Tmax

As can be seen in Tables 37 and 38, when test drug A (two tablets of Simvastatin 10 mg TCDS) was compared to the reference product (one tablet of Zocor 20 mg), the following values were obtained for Simvastatin levels:

The extent of absorption, as reflected by the AUC₍₀₋₃₆) has a ratio of 2.22 and a 90% ANOVA Confidence Interval of 1.73→2.83; The rate of absorption, as reflected by the Cmax values, has a ratio of 0.69 and a 90% ANOVA Confidence Interval of 0.47→1.02; The rate of absorption, as reflected by the Tmax values, has a Tmax difference of 3.83 hours with a range of 1.33 to 11.33; The rate of absorption, as reflected by the Lag-time values, has a Lag-time difference of 1.82 hours with a range of 1.33 to 2.17.

The following values were obtained for SHA levels:

The extent of absorption, as reflected by the AUC₍₀₋₃₆) has a ratio of 1.68 and a 90% ANOVA Confidence Interval of 1.39→2.04; The rate of absorption, as reflected by the Cmax values, has a ratio of 1.19 and a 90% ANOVA Confidence Interval of 0.94→1.50; The rate of absorption, as reflected by the Tmax values, has a Tmax difference of 4.21 hours with a range of −1.50 to 19.50; The rate of absorption, as reflected by the Lag-time values, has a Lag-time difference of 2.49 hours with a range of 1.50 to 4.17.

When test drug B (one tablet of Simvastatin 20 mg TCDS) was compared to the reference drug (one tablet of Zocor 20 mg), the following values were obtained for Simvastatin levels:

The extent of absorption, as reflected by the AUC₍₀₋₃₆) has a ratio of 2.00 and a 90% ANOVA Confidence Interval of 1.44→2.77; The rate of absorption, as reflected by the Cmax values, has a ratio of 0.71 and a 90% ANOVA Confidence Interval of 0.53→0.95; The rate of absorption, as reflected by the Tmax values, has a Tmax difference of 6.12 hours with a range of 2.00 to 14.50; The rate of absorption, as reflected by the Lag-time values, has a Lag-time difference of 1.82 hours with a range of 1.33 to 2.17.

The following values were obtained for SHA levels:

The extent of absorption, as reflected by the AUC₍₀₋₃₆) has a ratio of 1.33 and a 90% ANOVA Confidence Interval of 1.00→1.76; The rate of absorption, as reflected by the Cmax values, has a ratio of 0.99 and a 90% ANOVA Confidence Interval of 0.77→1.27; The rate of absorption, as reflected by the Tmax values, has a Tmax difference of 3.38 hours with a range of −0.50 to 11.50; The rate of absorption, as reflected by the Lag-time values, has a Lag-time difference of 2.49 hours with a range of 1.00 to 4.33.

When test drug A (two tablets of Simvastatin 10 mg TCDS) was compared to test drug B (one tablet of Simvastatin 20 mg TCDS), the following values were obtained for Simvastatin levels:

The extent of absorption, as reflected by the AUC(o-₃₆) has a ratio of 1.11 and a 90% ANOVA Confidence Interval of 0.92→1.34; The rate of absorption, as reflected by the Cmax values, has a ratio of 0.98 and a 90% ANOVA Confidence Interval of 0.70→1.39; The rate of absorption, as reflected by the Tmax values, has a Tmax difference of −2.29 hours with a range of −10.00 to 1.00; The rate of absorption, as reflected by the Lag-time values, has a Lag-time difference of 0.00 hours with a range of −0.50 to 0.50.

The following values were obtained for SHA levels:

The extent of absorption, as reflected by the AUC₍₀₋₃₆₎ has a ratio of 1.27 and a 90% ANOVA Confidence Interval of 0.98→1.65; The rate of absorption, as reflected by the Cmax values, has a ratio of 1.20 and a 90% ANOVA Confidence Interval of 0.91→1.59; The rate of absorption, as reflected by the Tmax values, has a Tmax difference of 0.83 hours with a range of −10.00 to 19.50; The rate of absorption, as reflected by the Lag-time values, has a Lag-time difference of 0.00 hours with a range of −1.50 to 1.50.

These results indicate a higher bioavailability of simvastatin (about two fold) when comparing Test drug A (two simvastatin 10 mg TCDS) and Test drug B (one simvastatin 20 mg TCDS) to the reference drug (one simvastatin 20 mg IR), with AUC₍₀₋₃₆₎ ratios of 2.22 and 2.00, respectively.

In addition, the results indicate 30% reduction in maximal concentration (Cmax) of simvastatin when comparing Test drug A and Test drug B to the reference drug, with Cmax ratios of 0.69 and 0.71, respectively.

The Lag Time for simvastatin was delayed by 1.82 h for both Test drug A and Test drug B in comparison to the reference drug, and the time to reach maximal concentration (Tmax) was delayed in 3.83 h and 6.12 h, respectively in comparison with the reference drug.

The results further indicate a higher bioavailability of the active metabolite SHA (about 1.5 fold) when comparing Test drug A and Test drug B to the reference drug, with AUC₍₀₋₃₆₎ ratios of 1.68 and 1.33, respectively.

In addition, the results indicate approximately similar maximal concentration (Cmax) of SHA when comparing Test drug A and Test drug B to the reference drug, with Cmax ratios of 1.19 and 0.99, respectively.

The Lag Time for SHA was delayed by 2.49 h for both Test drug A and Test drug B in comparison to the reference drug, and the time to reach maximal concentration (Tmax) was delayed by 4.21 h and 3.38 h, respectively in comparison to the reference drug.

The pharmacokinetic parameters for both the parent compound (simvastatin) and the active metabolite (SHA) show approximately similar results when comparing Test drug A to Test drug B.

Therefore, lower doses of the test formulation can be used for achieving the designed efficacy with a better safety profile.

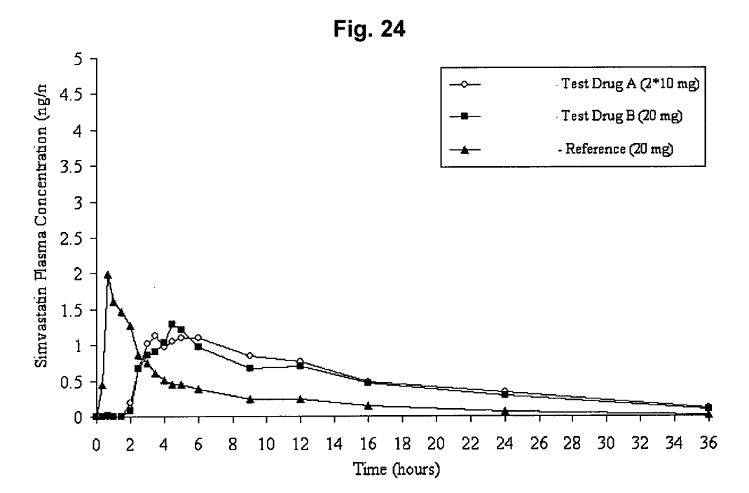

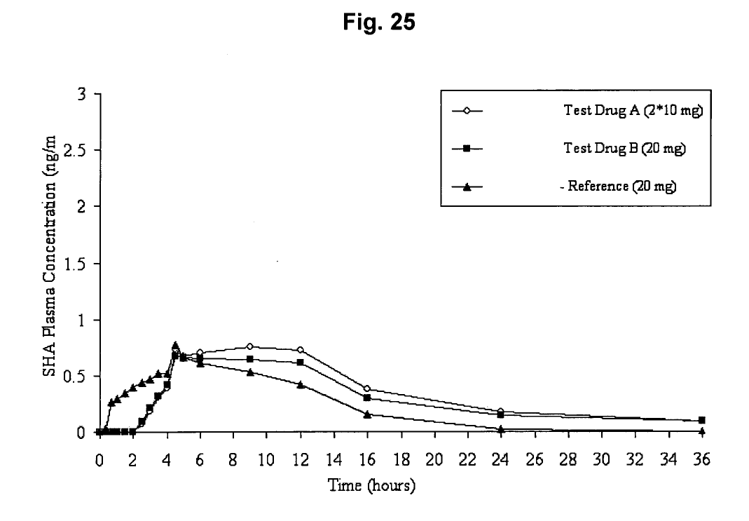

Table 39 and 40, and FIGS. 24 and 25 herein, present mean plasma levels of simvastatin and of the active metabolite SHA, respectively.

TABLE 39
Mean simvastatin plasma concentrations:

| | Simvastatin | Simvastatin | |
|---|---|---|---|
| | 10 mg TCDS | 20 mg TCDS | Zocor 20 mg |
| | Two Tablets | One Tablet | One Tablet |
| TIME | (DPT) | (DPT) | (MSD) |
| (hours) | (ng/ml) | (ng/ml) | (ng/ml) |
| 0.00 | BLQ | BLQ | BLQ |
| 0.33 | BLQ | BLQ | 0.446 |
| 0.67 | BLQ | 0.011* | 1.99 |
| 1.00 | BLQ | BLQ | 1.61 |
| 1.50 | BLQ | BLQ | 1.47 |
| 2.00 | 0.185 | 0.075* | 1.27 |
| 2.50 | 0.662 | 0.672 | 0.867 |
| 3.00 | 1.01 | 0.867 | 0.743 |
| 3.50 | 1.13 | 0.909 | 0.608 |
| 4.00 | 0.969 | 1.03 | 0.503 |
| 4.50 | 1.05 | 1.29 | 0.450 |
| 5.00 | 1.11 | 1.21 | 0.445 |
| 6.00 | 1.10 | 0.967 | 0.379 |
| 9.00 | 0.840 | 0.662 | 0.238 |
| 12.00 | 0.767 | 0.696 | 0.238 |
| 16.00 | 0.483 | 0.458 | 0.140 |
| 24.00 | 0.337 | 0.293 | 0.068* |
| 36.00 | 0.110 | 0.095* | 0.009* |

BLQ: Below Limit of Quantification
*The mean value is BLQ

TABLE 40
Mean SHA plasma concentrations:

| | Simvastatin | Simvastatin | |
|---|---|---|---|
| | 10 mg TCDS | 20 mg TCDS | Zocor 20 mg |
| | Two Tablets | One Tablet | One Tablet |
| TIME | (DPT) | (DPT) | (MSD) |
| (hours) | (ng/ml) | (ng/ml) | (ng/ml) |
| 0.00 | BLQ | BLQ | BLQ |
| 0.33 | BLQ | BLQ | 0.034* |
| 0.67 | BLQ | BLQ | 0.259 |
| 1.00 | BLQ | BLQ | 0.297 |
| 1.50 | BLQ | BLQ | 0.348 |
| 2.00 | BLQ | BLQ | 0.397 |
| 2.50 | 0.069* | 0.089* | 0.438 |
| 3.00 | 0.181 | 0.214 | 0.469 |
| 3.50 | 0.304 | 0.320 | 0.517 |
| 4.00 | 0.384 | 0.417 | 0.522 |
| 4.50 | 0.737 | 0.667 | 0.771 |
| 5.00 | 0.672 | 0.662 | 0.665 |
| 6.00 | 0.705 | 0.655 | 0.608 |
| 9.00 | 0.752 | 0.645 | 0.529 |
| 12.00 | 0.725 | 0.608 | 0.414 |
| 16.00 | 0.372 | 0.298 | 0.157 |
| 24.00 | 0.174 | 0.141 | 0.021* |
| 36.00 | 0.089* | 0.087* | BLQ |

BLQ: Below Limit of Quantification
*The mean value is BLQ

FIG. 24 illustrates mean plasma simvastatin concentration-time curves.

FIG. 25 illustrates mean plasma SHA concentration-time curves.

In FIGS. 24 and 25, empty diamonds represent the mean plasma levels obtained with Test drug A, full squares represent the mean plasma levels obtained with Test drug B and full triangles represent the mean plasma levels obtained with the Reference drug.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1.-37. (canceled)

38. A delayed burst release oral formulation for localized release of a statin or a pharmaceutically acceptable salt or ester thereof in the gastrointestinal tract of a subject, comprising
a. a core comprising at least one statin, and at least one burst controlling agent, wherein the burst controlling agent is a water insoluble polymer;
b. a subcoat surrounding the core comprising at least one water soluble hydrophilic carrier; and
c. an outer coating over the core, the outer coating comprising a water insoluble hydrophobic carrier and a water insoluble hydrophilic particulate matter, the water insoluble hydrophilic particulate matter allowing entry of liquid into the core.

39. The formulation of claim 38, wherein the outer coating comprises a combination of at least one swellable polymer and at least one water insoluble polymer selected from the group consisting of a cross-linked polysaccharide, a water insoluble starch, microcrystalline cellulose, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, and cross-linked polyacrylic acid.

40. The formulation of claim 38, wherein the outer coating is a two-layered coating comprising a rupturing outer layer and swellable inner layer.

41. The formulation of claim 38, wherein the outer coating retains less than 2% of the total amount of the statin in the formulation, as measured in vitro following fast disintegration of a split dosage form, or wherein the outer coating retains less than 1% of the total amount of the statin in the formulation, as measured in vitro following fast disintegration of a split dosage form.

42. The formulation of claim 38, wherein the water soluble hydrophilic carrier of the subcoat is selected from the group consisting of povidone (PVP: polyvinyl pyrrolidone), polyvinyl alcohol, copolymer of PVP and polyvinyl acetate, hydroxypropyl cellulose (HPC), hydroxypropyl methylcellulose HPMC, carboxy methyl cellulose, hydroxyethyl cellulose, gelatin, polyethylene oxide, acacia, dextrin, magnesium aluminum silicate, starch, polyacrylic acid, polyhydroxyethylmethacrylate (PHEMA), polymethacrylates and copolymers thereof, gum, water soluble gum, polysaccharide, hydroxypropylmethyl cellulose phthalate, polyvinyl acetate phthalate, cellulose acetate phthalate ,hydroxypropylmethyl cellulose acetate succinate, poly(methacrylic acid, methyl methacrylate)1:1 and poly(methacrylic acid, ethyl acrylate)1:1, alginic acid, and sodium alginate, and any other pharmaceutically acceptable polymer that dissolves in phosphate buffer pH>5.5 or mixtures thereof.

43. The formulation of claim 42, wherein the water soluble hydrophilic carrier is polyvinyl pyrrolidone.

44. The formulation of claim 38, wherein the subcoat further comprises at least one water insoluble particulate matter selected from the group consisting of microcrystalline cellulose, ethylcellulose, a cross-linked polysaccharide, a water insoluble starch, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, talc, silicon dioxide and cross-linked polyacrylic acid.

45. The formulation of claim 44, wherein the water insoluble particulate matter is microcrystalline cellulose.

46. The formulation of claim 38, wherein the water insoluble hydrophilic particulate matter forms channels in the outer coating upon contact with a liquid, whereby the channels absorb the liquid and cause the at least one burst controlling agent to burst the coating, thereby providing delayed burst release of the statin, so that the formulation releases substantially no statin in vitro for at least about 1 to 1.5 hours, and then releases at least about 60% of the statin in vitro about 1 hour after the delayed burst release occurs, with the release of the statin occurring for at least 12 hours.

47. The formulation of claim 38, wherein the statin is selected from the group consisting of simvastatin, lovastatin, mevastatin, pravastatin, fluvastatin, atorvastatin, pitavastatin and rivastatin.

48. The formulation of claim 38, wherein the outer coating further comprises one or more of a surfactant, at least one disintegrant, or an enteric coating disposed over the outer coating.

49. The formulation of claim 48, wherein the surfactant, when present, is sodium lauryl sulfate (SLS), the disintegrant, when present, is croscarmellose sodium, and the enteric coating, when present, comprises a methacrylic acid copolymer, and optionally further comprises a plasticizer.

50. The formulation of claim 38, wherein the water soluble hydrophilic carrier of the subcoat is a combination of povidone and microcrystalline cellulose.

51. The formulation of claim 38, wherein the water insoluble polymer of the core is selected from the group consisting of a cross-linked polysaccharide, a water insoluble starch, microcrystalline cellulose, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, and cross-linked polyacrylic acid.

52. The formulation of claim 49, wherein the water insoluble polymer is talc, microcrystalline cellulose or a combination thereof.

53. The formulation of claim 49, wherein the water-insoluble hydrophobic carrier of the outer coating is ethylcellulose; or the water insoluble hydrophilic particular matter of the outer coating is microcrystalline cellulose.

54. The formulation of claim 38, wherein the water soluble hydrophilic polymer is povidone K and the subcoat further comprises microcrystalline cellulose PH-101, and wherein the water insoluble particulate matter is microcrystalline cellulose PH-102, and the outer coating further comprises ethyl cellulose and cetyl alcohol, and the formulation optionally comprises a surfactant.

55. The formulation of claim 38 for release of a statin or a pharmaceutically acceptable salt or ester thereof mainly in the colon of a subject, comprising:
(a) a core that comprises an effective amount of statin or a pharmaceutically acceptable salt or ester thereof wherein the core contains at least one burst controlling agent and at least one disintegrant, and wherein the core is formed as a compressed tablet;
(b) a subcoat surrounding the core comprising at least one water soluble hydrophilic carrier; and
(c) an outer coating over the core, the outer coating comprising a water insoluble hydrophobic carrier and a water-insoluble but hydrophilic particulate matter, contained in the carrier, that forms channels in the outer coating material upon contact with the colon medium, wherein the channels imbibe liquid and cause the at least one burst controlling agent to burst the coating, thereby providing delayed burst release of the statin or a pharmaceutically acceptable salt or ester thereof after at least two hours providing pharmacologically effective blood levels over a period extending over at least about 12 hours.

56. The formulation of claim 55, wherein the core further comprises colloidal silicone dioxide.

57. A method for providing a therapeutically effective amount of a statin, a pharmaceutically acceptable salt or ester thereof or an active form thereof to a subject, comprising orally administering to the subject a delayed burst release formulation according to claim 38.

58. A method for providing enhanced bioavailability of a statin, a pharmaceutically acceptable salt or ester thereof or an active form thereof to the circulation of a subject, as measured by the AUC compared to a substantially similar dose of an immediate release formulation of the statin, comprising orally administering to the subject a delayed burst release formulation according to claim 38.

59. A method of providing a delayed fast release of a statin, a pharmaceutically acceptable salt or ester thereof or an active form thereof in the gastrointestinal tract of a subject, comprising orally administering to the subject a delayed burst release formulation according to claim 38.
